(19) United States
(12) Patent Application Publication
(10) Pub. No.: US 2023/0248745 A1
(43) Pub. Date: Aug. 10, 2023

(54) SILVER ENHANCED CANNABINOID ANTIBIOTICS

(71) Applicant: The University of British Columbia, Vancuver British Columbia (CA)

(72) Inventors: John K. Jackson, West Vancouver, British Columbia (CA); Charles J. Thompson, Vancouver, British Columbia (CA); Dana M. Lambert, Vancouver, British Columbia (CA); Manisha Dosanjh, Surrey, British Columbia (CA)

(21) Appl. No.: 18/015,159

(22) PCT Filed: Jul. 20, 2021
(86) PCT No.: PCT/CA2021/051005

§ 371 Date: Jan. 9, 2023

Related U.S. Application Data
(60) Provisional application No. 63/055,211, filed on Jul. 22, 2020.
(60) Provisional application No. 63/111,972, filed on Nov. 10, 2020.

(51) Int. Cl. A61K 31/00; A61K 33/38; A61P 31/04

(52) U.S. Cl. CPC A61K 31/658; A61K 33/38; A61P 31/04

(57) ABSTRACT

Pharmaceutical formulations and treatments are provided that make combined use of selected antibiotic cannabinoids with silver-containing medicaments. Therapeutically effective regimens are provided that facilitate positive drug-drug interactions between the cannabinoid and the silver-containing medicament in a subject.

### FIELD OF THE INVENTION

The invention is in the field of medicinal preparations and treatments involving the combined use of silver-containing medicaments and specific phenolic cannabinoids.

### BACKGROUND OF THE INVENTION

A very wide range of physiological activities have been ascribed to compounds derived from flowering plants in the genus Cannabis, particularly phytocannabinoid compounds (see Cunha et al., 1980; Morales et al., 2017; U.S. Pat. No. 6,630,507). There are more than 80 cannabinoids found in cannabis plant extracts (Russo, 2011), including: cannabidiol (CBD), its acid form cannabidiolic acid (CBDA), cannabichromene (CBC), its acid form cannabichromenic acid (CBCA), cannabigerol (CBG), its acidic form cannabigerolic acid (CBGA), tetrahydrocannabinol (THC), and its acidic form, tetrahydrocannabinolic acid (THCA). Studies have suggested that Cannabis extracts, or compounds derived from the Cannabis plant, have a very wide range of, often ill defined, anti-microbial activities (Van Klingeren & Ten Ham, 1976; Abdelaziz, 1982; Appendino et al., 2011; Appendino et al., 2008; Eisohly et al., 1982; Eisohly et al., 1982; Appendino et al., 2008; Turner & Eisohly, 1981; Mechoulam & Gaoni, 1965; WO2012/012498; WO2018/011813).

Silver in a variety of chemical forms has an ancient history as an antiseptic, with antimicrobial medicinal properties of silver for example being described by Hippocrates. More recently, with the invention of potent antibiotics in the 20th century, beginning with sulfa drugs and penicillin, the use of silver as an antimicrobial has assumed less clinical significance. Antimicrobial uses of silver compounds nevertheless remain important, and a variety of silver nanoparticles have relatively recently been added to the catalogue of silver antimicrobials, a catalogue which includes metallic silver, silver nitrate, silver sulfate, silver oxide, silver chloride, silver lactate and silver sulfadiazine (Rai et al., 2009; Khundkar et al., 2010; Barnea et al., 2010; Franci et al, 2015). Colloidal silver is a term used for a category of commercial products, frequently characterized as suspensions of silver-containing particles between 1 and 1000 nm in size, in formulations that may also contain a number of other forms of silver such as silver ions, nanoscale silver oxide, silver chloride, silver sulfide, or metallic silver (along with stabilizers and additives). Silver nanoparticles, for purposes of the present application, are particles of silver between about 1 nm and 100 nm in size, comprised principally of metallic silver and/or silver oxide. Silver nanoparticles have been described as improving the antibiotic effect of ampicillin, chloramphenicol and kanamycin against gram positive and gram negative bacteria (Hwang et al., 2012). Similarly, silver nitrate has been described as enhancing the antibiotic action of ampicillin, gentamicin and olfoxacin against gram negative bacteria, and sensitizing gram negative bacteria to gram-positive-specific antibiotics such as vancomycin (Ruben Morones-Ramirez et al., 2013). Clinicians rely upon silver-containing wound care products and medical devices as an alternative to other antibiotics because of the increase in antibiotic-resistant bacteria and the resultant reduction in first-line antibiotic prescribing (Gemmell et al. 2006, Chopra 2007). However, clinical evidence indicates a lack of efficacy of currently used silver-containing products in preventing catheter-associated urinary tract infections (Lam et al. 2014), treating infected wounds (Vermeulen et al. 2007), preventing infection in burns and other wounds (Storm-Versloot et al. 2010) and treating diabetic ulcers (Bergin et al. 2006). Furthermore, the use of silver-containing products in medical practice has been associated with the emergence of silver-resistant bacteria (Hosny et al. 2019). Bacterial resistance to silver was first reported in 1975 when McHugh and colleagues described a silver-resistant strain of Salmonella typhimurium isolated from a hospital burns unit which led to an outbreak and three cases of patient mortality (McHugh et al. 1975). Several studies have since identified the molecular mechanisms involved in silver resistance, which can be of endogenous mutational (Lok et al. 2008, Finley et al. 2015, Staehlin et al. 2016, Massani et al. 2018, Hanczvikkel et al. 2018) or exogenous horizontally-acquired origin (Gupta et al. 1999, Sutterlin et al. 2014, Fang et al. 2016). Strategies to improve the efficacy of silver-containing antimicrobials and minimize the emergence of bacterial resistance are clearly needed. Experts recommend silver-containing antimicrobials should provide rapid bactericidal activity in order to promote efficacy and limit overall bacterial exposure thereby preventing the development of resistance (Chopra 2007).

### BRIEF DESCRIPTION OF THE DRAWINGS

FIG. 1 includes 3 line graphs, illustrating the results of MRSA kill-curve tests with CBG and AgNO3.

FIG. 2 includes 3 line graphs, illustrating the results of MRSA kill-curve tests with CBC and AgNO3.

FIG. 3 includes 3 line graphs, illustrating the results of MRSA kill-curve tests with CBC and AgNP.

FIG. 4 includes 4 photographs, illustrating the results of MRSA agar plate test with CBGA at sub-MICs and different AgNP concentrations.

FIG. 5 is an annotated photograph, illustrating the antibiotic effect of combinations of the indicated cannabinoids and the indicated silver-containing antibiotics in: the first row of plates—PVA films; and, in the second row of plates—catheters coated with PVA films.

FIG. 6 includes 3 line graphs, illustrating the results of MRSA kill-curve tests with CBGA and AgNP.

FIG. 7 includes 3 line graphs, illustrating the results of MRSA kill-curve tests with CBG and AgNP.

FIG. 8 includes 2 line graphs, illustrating the results of MRSA kill-curve tests with CBD and AgNP.

FIG. 9 includes 2 line graphs, illustrating the results of MRSA kill-curve tests with CBCA and AgNP.

FIG. 10 includes 9 line graphs, illustrating the results of E. coli kill curve tests with cannabinoids and silver sulfate.

### SUMMARY

One general aspect of the innovations disclosed herein includes methods of treating or preventing a bacterial infection in a subject in need thereof. The method of treating or preventing involves the administration of a cannabinoid that is one or more of cannabidiol (CBD), cannabidiolic acid (CBDA), cannabigerol (CBG), cannabigerolic acid (CBGA), cannabichromenic acid (CBCA) or cannabichromene (CBC); and a silver-containing medicament. The cannabinoid and the silver-containing medicament may each be administered in a regimen, and the combination of the regimens adapted to provide a positive drug-drug interaction between the cannabinoid and the silver-containing medicament in the subject.

Implementations may include one or more of the following features. The method where the positive drug-drug interaction between the cannabinoid and the silver-containing medicament is a positive antibiotic drug-drug interaction that enhances the antibiotic effect of the cannabinoid and/or the silver-containing medicament in the subject. The positive drug-drug antibiotic interaction may for example include a synergistically effective combined antibiotic activity. The bacterial infection may be an infection by a gram positive and/or gram negative bacteria, such as a plurality of gram positive and/or gram negative bacteria. The bacterial infection may be an infection by an antibiotic resistant bacteria.

The cannabinoid may be administered in a regimen that reduces the minimum inhibitory concentration (MIC) of the silver-containing medicament. The cannabinoid may for example reduce the MIC of the silver-containing medicament when the cannabinoid is present in an amount that is less than the MIC of the cannabinoid. The silver-containing medicament may be administered in a regimen that reduces the MIC of the cannabinoid. The silver-containing medicament may reduce the MIC of the cannabinoid when the silver-containing medicament is present in an amount that is less than the MIC of the silver-containing medicament.

The cannabinoid may be administered in a relative amount that provides at least a 2 to 128 fold decrease in the MIC of the silver-containing medicament. The silver-containing medicament may be administered in a relative amount that provides at least a 2 to 128 fold decrease in the MIC of the cannabinoid. The cannabinoid may be one of CBD, CBDA, CBG, CBGA, CBCA or CBC. The cannabinoid may be two, three, four or five of CBD, CBDA, CBG, CBGA, CBCA or CBC. The cannabinoid may be all six of CBD, CBDA, CBG, CBGA, CBCA and CBC. The cannabinoid may for example be derived from a plant, such as Cannabis sativa or Cannabis indica.

In select embodiments, no antibiotic other than the cannabinoid and the silver-containing medicament is administered to the subject. The method may include administering to the subject the effective amounts of the cannabinoid and the silver-containing medicament, with no other medicaments, or no other antibiotics, or where no phytocannabinoid other than the cannabinoid is administered to the subject. The silver-containing medicament may be one or more of: a silver salt, silver nitrate, silver sulfate, silver oxide, silver chloride, silver lactate, a silver nanoparticle, a colloidal silver, a silver zeolite, or silver sulfadiazine. The subject may be a mammal, such as a human patient.

The therapeutically effective regimen of the cannabinoid may include administration of from 0.001 to 5,000 mg per day of the cannabinoid. The therapeutically effective regimen of the silver-containing medicament may include administration of from 0.001 to 10,000 mg per day elemental silver of the silver-containing medicament. The cannabinoid and the silver-containing medicament may be co-administered. The cannabinoid and the silver-containing medicament may be administered sequentially, in any order.

One general aspect includes an antibiotic formulation that includes a cannabinoid that is one or more of cannabidiol (CBD), cannabidiolic acid (CBDA), cannabigerol (CBG), cannabigerolic acid (CBGA), cannabichromenic acid (CBCA) or cannabichromene (CBC); and a silver-containing medicament. In the antibiotic formulation, the cannabinoid and the silver-containing medicament may each be present in an amount, and the combination of the amounts provides a positive drug-drug interaction between the cannabinoid and the silver-containing medicament in a subject when the formulation is administered to the subject.

Implementations may include one or more of the features summarized above, in the discussion of the therapeutic regimen, or as follows. In the antibiotic formulation, the cannabinoid may for example be present at 0.01-5% w/w. The silver-containing medicament may be present in the formulation at 0.01-5% w/w. The cannabinoid and/or the silver-containing medicament may be dissolved, dispersed, mixed or suspended in the formulation with a pharmaceutically acceptable carrier.

The positive drug-drug interaction between the cannabinoid and the silver-containing medicament may be a positive antibiotic drug-drug interaction that enhances the antibiotic effect of the cannabinoid and/or the silver-containing medicament in the subject. The positive drug-drug antibiotic interaction may include a synergistically effective combined antibiotic activity. The cannabinoid may be one, two, three, four or five of CBD, CBDA, CBG, CBGA, CBCA or CBC, or the cannabinoid may be all six of CBD, CBDA, CBG, CBGA, CBCA and CBC. The cannabinoid may be derived from a plant, such has a Cannabis sativa or Cannabis indica plant.

In some embodiments, no antibiotic other than the cannabinoid and the silver-containing medicament is present in the formulation. The formulation may be made up essentially of the cannabinoid and the silver-containing medicament as active ingredients, i.e. including no other active medicament ingredients. The formulation may for example include no phytocannabinoid other than the cannabinoid. The silver-containing medicament may be one or more of: a silver salt, silver nitrate, silver sulfate, silver oxide, silver chloride, silver lactate, a silver nanoparticle, a colloidal silver, a silver zeolite, or silver sulfadiazine.

The antibiotic formulation may be for use in formulating a medicament for treating or preventing a bacterial infection in a subject in need thereof, as summarized above.

The antibiotic formulation may be provided in or coating a supporting matrix, such as a gel, a hydrogel, a film, a polymer or a ceramic. The antibiotic formulation may be a hydrogel formulation that is a dried film. The dried film may be in the form of, or is for use as, a wound dressing. The hydrogel material may for example be a polyvinyl alcohol (PVA). The hydrogel formulation may for example coat a catheter, such as a urethral catheter. The supporting matrix may be in the form of a wound dressing, a biomedical implant, an endotracheal tube, a surgical mask, cotton fibers, synthetic fibers, a component of an invasive medical device, a catheter or a catheter coating. The matrix may include more than one silver-containing medicament and the releasability of the different silver-containing medicaments from the matrix may be different. For example, a first silver-containing medicament may be formulated in the matrix for sustained release, and a second silver-containing medicament formulated in the matrix for quick release. The matrix may include one or more additional medicaments, and the releasability from the matrix of the additional medicament(s) may be different from the releasability of the silver-containing medicament. The additional medicament may fore example be an additional antibiotic.

### DETAILED DESCRIPTION OF THE INVENTION

In one aspect, pharmaceutical formulations and treatments are provided that make combined use of selected antibiotic cannabinoids with silver-containing medicaments. In particular, formulations may include cannabidiol (CBD), cannabidiolic acid (CBDA), cannabigerol (CBG), cannabigerolic acid (CBGA), cannabichromenic acid (CBCA) and/or cannabichromene (CBC). Therapeutically effective regimens are provided that facilitate positive drug-drug interactions between the cannabinoid and the silver-containing medicament in a subject. In select embodiments, these positive drug-drug interactions may provide antibiotic synergy.

The antibiotically effective ingredients may for example be provided in synergistically effective relative amounts. For example, the cannabinoid and the silver-containing medicament may be provided at concentrations that are only antibiotically active in synergistic combinations, such as 1, 2, 3 or 4 μg/ml of the cannabinoid. In synergistic combination, the inhibitory concentrations of the cannabinoid and/or the silver-containing medicament may for example decrease, for example by two or more fold, for example from 2-16 fold. Alternatively, the relative weight ratio of cannabinoid to silver-containing medicament may for example be from about 4:1 to 1:16.

In select embodiments, synergies and/or potentiation effects are maximized using concentrations of antibiotically active components that are below the MICs for each component, for example just below the MICs. The components may accordingly be present in relative amounts that approximate the ratio of the respective MICs for the components. For example, this may occur when the molar ratio of silver-containing medicament:cannabinoid is from 1:100 to 100:1 (reflecting the MIC ratio of the components) 3:1 to 24:1.

Anti-microbial formulations may be used to prophylactically or therapeutically treat microbial infections, or otherwise inhibit microbial growth or multiplication. An antibiotic is an antimicrobial that is active against bacteria, and in this context includes naturally-occurring, semi-synthetic and synthetic substances that kill or inhibit the growth or multiplication of bacteria by any mechanism, including antiseptic or disinfectant modalities.

Subjects amenable to treatment include mammalian subjects, such as human patients, laboratory animals (e.g., primates, rats, mice), livestock (e.g., cows, sheep, goats, pigs, horses, fowl), or household pets (e.g., dogs, cats, rodents, birds), for example belonging to the taxonomic groups of primates, canines, felines, bovines, caprines, equines, ovines, porcines, rodents, Ayes or lagomorphs. Human patients to be treated may for example be male or female, or at a specific stage of development: neonate, infant, juvenile, adolescent, adult and geriatric. Specific veterinary indications amenable to treatment may for example include enterococcal infections in poultry, for example treatment of Enterococcus cecorum infections in chickens.

The cannabinoid may for example be obtained from a plant extract, such as an extract of Cannabis sativa or Cannabis indica. A wide variety of methods may be used to prepare these plant extracts, including, but not limited to, supercritical or subcritical extraction with CO₂, extraction with hot gas, and extraction with solvents. Biosynthetic approaches to the production of cannabinoids are also available, as are a variety of synthetic approaches (based for example on approaches used to synthesize THC/dronabinol, see U.S. Pat. No. 7,323,576 and Trost and Dogra, 2007). Alternative approaches involve expressing cannabinoid biosynthetic genes in recombinant hosts, such as recombinant yeast (see Luo et al., 2019). The cannabinoid components of the formulation may accordingly be from a culture, such as a culture of a recombinant host, such as a recombinant yeast expressing the components. Formulations may also specifically exclude additional cannabinoids, terpenoids or terpenes, including plant-derived phytocannabinoids, terpenoids or terpenes, such as astaxanthin or other sesquiterpenes, tetraterpenes, triterpenes, diterpenes or monoterpenes. Alternatively, one or more additional compounds may be included, or specifically excluded, in alternative formulations, including for example: terpenes, terpenoid, sterols, triglycerides, alkanes, squalene, tocopherol, carotenoids, chlorophyll, flavonoid glycosides, or alkaloids.

A titratable dosage may for example be adapted to allow a patient to take the medication in doses smaller than the unit dose, wherein a “unit dose” is defined as the maximum dose of medication that can be taken at any one time or within a specific dosage period. Titration of doses will allow different patients to incrementally increase the dose until they feel that the medication is efficacious, as not all patients will require the same dose to achieve the same benefits. A person with a larger build or faster metabolism may require larger doses to achieve the same effect as another with a smaller build or slower metabolism. Therefore, a titratable dosage has advantages over a standard dosage form.

In select embodiments, formulations may be adapted to be delivered in such a way as to target one or more of the following: dental, sublingual, buccal, oral, rectal, nasal, vaginal, parenteral and via the pulmonary system. Formulations may for example be in one or more of the following forms: gel, gel spray, tablet, liquid, capsule, by injection, or for vaporization.

Conventional pharmaceutical practice may be employed to provide suitable formulations or compositions to administer the formulations to subjects. Routes of administration may for example include, parenteral, intravenous, intradermal, subcutaneous, intramuscular, intracranial, intraorbital, ophthalmic, intraventricular, intracapsular, intraspinal, intrathecal, intracisternal, intraperitoneal, intranasal, inhalational, aerosol, topical, sublingual or oral administration. Therapeutic formulations may be in the form of liquid solutions or suspensions; for oral administration, formulations may be in the form of tablets or capsules; for intranasal formulations, in the form of powders, nasal drops, or aerosols; and for sublingual formulations, in the form of drops, aerosols or tablets. Formulations may be presented inside or as coatings on devices such as (but not restricted to) bone cement, dental cement, dental implants, wound dressings, catheter lines, injectable pastes or microimplants. In certain embodiments wound dressings may be manufactured from polymers such as poly vinyl alcohol or from numerous hydrogel forming materials such as (but not limited to) alginate/calcium, hyaluronic acid, cellulose derivatives, poloxamers and carbomers, pegylated polymers, chitosan or combinations of these or from materials well known to pharmaceutical scientists and outlined by Kamoun E et al (2017) in either a solvent cast or electrospun membrane form. Materials may be used as described or further modified chemically to improve performance. Alternatively, existing, commercially available, wound dressings may be simply soaked in any of the formulations. Implants may be simply coated with the drug formulations directly or provided in a coating material that both anchors the drugs to the implants whilst potentially providing controlled release of the drugs. Implant coating materials may be polymeric, ceramic, ionic, metals, paint-like materials or hydrogels.

Methods well known in the art for making formulations are found in, for example, “Remington: The Science and Practice of Pharmacy” (21st edition), ed. David Troy, 2006, Lippincott Williams & Wilkins. Formulations for parenteral administration may, for example, contain excipients, sterile water, or saline, polyalkylene glycols such as polyethylene glycol, oils of vegetable origin, or hydrogenated napthalenes. Numerous polymeric systems may be used to encapsulate the drugs to provide both a suitable means of drug administration and/or a controlled release aspect. Systems may be presented as monolithic units such as films or seeds or as microspheres, pastes, gels, nanoparticles. These systems may be manufactured from numerous degradable or non degradable polymers which are well described by Leichty W et al 2017. Other potentially useful parenteral delivery systems include osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Pharmaceutical compositions of the present invention may be in any form which allows for the composition to be administered to a patient. For example, the composition may be in the form of a solid, liquid or gas (aerosol). Pharmaceutical compositions of the invention are formulated so as to allow the active ingredients contained therein to be bioavailable upon administration of the composition to a patient. Compositions that will be administered to a patient may take the form of one or more dosage units, where for example, a tablet, capsule or cachet may be a single dosage unit, and a container of the compound in aerosol form may hold a plurality of dosage units.

Materials used in preparing the pharmaceutical compositions should be pharmaceutically pure and non-toxic in the amounts used. The inventive compositions may include one or more compounds (active ingredients) known for a particularly desirable effect. It will be evident to those of ordinary skill in the art that the optimal dosage of the active ingredient(s) in the pharmaceutical composition will depend on a variety of factors. Relevant factors include, without limitation, the type of subject (e.g., human), the particular form of the active ingredient, the manner of administration and the composition employed.

In general, the pharmaceutical composition includes a formulation of the present invention as described herein, in admixture with one or more carriers. The carrier(s) may be particulate, so that the compositions are, for example, in tablet or powder form. The carrier(s) may be liquid, with the compositions being, for example, an oral syrup or injectable liquid. In addition, the carrier(s) may be gaseous, so as to provide an aerosol composition useful in, e.g., inhalatory administration.

When intended for oral administration, the composition is preferably in either solid or liquid form, where semi-solid, semi-liquid, suspension and gel forms are included within the forms considered herein as either solid or liquid.

As a solid formulation for oral administration, the composition may be formulated into a powder, granule, compressed tablet, pill, capsule, cachet, chewing gum, wafer, lozenges, or the like form. Such a solid composition will typically contain one or more inert diluents or edible carriers. In addition, one or more of the following adjuvants may be present: binders such as syrups, acacia, sorbitol, polyvinylpyrrolidone, carboxymethylcellulose, ethyl cellulose, microcrystalline cellulose, gum tragacanth or gelatin, and mixtures thereof; excipients such as starch, lactose or dextrins, disintegrating agents such as alginic acid, sodium alginate, Primogel, corn starch and the like; lubricants such as magnesium stearate or Sterotex, fillers such as lactose, mannitols, starch, calcium phosphate, sorbitol, methylcellulose, and mixtures thereof; lubricants such as magnesium stearate, high molecular weight polymers such as polyethylene glycol, high molecular weight fatty acids such as stearic acid, silica, wetting agents such as sodium lauryl sulfate, glidants such as colloidal silicon dioxide; sweetening agents such as sucrose or saccharin, a flavoring agent such as peppermint, methyl salicylate or orange flavoring, and a coloring agent. When the composition is in the form of a capsule, e.g., a gelatin capsule, it may contain, in addition to materials of the above type, a liquid carrier such as polyethylene glycol or a fatty oil.

The formulation may be in the form of a liquid, e.g., an elixir, syrup, solution, aqueous or oily emulsion or suspension, or even dry powders which may be reconstituted with water and/or other liquid media prior to use. The liquid may be for oral administration or for delivery by injection, as two examples. When intended for oral administration, preferred compositions contain, in addition to the present compounds, one or more of a sweetening agent, thickening agent, preservative (e.g., alkyl p-hydoxybenzoate), dye/colorant and flavor enhancer (flavorant). In a composition intended to be administered by injection, one or more of a surfactant, preservative (e.g., alkyl p-hydroxybenzoate), wetting agent, dispersing agent, suspending agent (e.g., sorbitol, glucose, or other sugar syrups), buffer, stabilizer and isotonic agent may be included. The emulsifying agent may be selected from lecithin or sorbitol monooleate.

The liquid pharmaceutical formulations of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

The pharmaceutical formulation may be intended for topical administration, in which case the carrier may suitably comprise a solution, emulsion, ointment, cream or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The formulation may be intended for rectal administration, in the form, e.g., of a suppository which will melt in the rectum and release the drug. The composition for rectal administration may contain an oleaginous base as a suitable nonirritating excipient. Such bases include, without limitation, lanolin, cocoa butter and polyethylene glycol. Low-melting waxes are preferred for the preparation of a suppository, where mixtures of fatty acid glycerides and/or cocoa butter are suitable waxes. The waxes may be melted, and the aminocyclohexyl ether compound is dispersed homogeneously therein by stirring. The molten homogeneous mixture is then poured into convenient sized molds, allowed to cool and thereby solidify.

The formulation may include various materials which modify the physical form of a solid or liquid dosage unit. For example, the composition may include materials that form a coating shell around the active ingredients. The materials which form the coating shell are typically inert, and may be selected from, for example, sugar, shellac, and other enteric coating agents. Alternatively, the active ingredients may be encased in a gelatin capsule or cachet.

The pharmaceutical formulation may consist of gaseous dosage units, e.g., it may be in the form of an aerosol. The term aerosol is used to denote a variety of systems ranging from those of colloidal nature to systems consisting of pressurized packages. Delivery may be by a liquefied or compressed gas or by a suitable pump system which dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit.

Some biologically active compounds may be in the form of the free base or in the form of a pharmaceutically acceptable salt such as the hydrochloride, sulfate, phosphate, citrate, fumarate, methanesulfonate, acetate, tartrate, maleate, lactate, mandelate, salicylate, succinate and other salts known in the art. The appropriate salt would be chosen to enhance bioavailability or stability of the compound for the appropriate mode of employment (e.g., oral or parenteral routes of administration).

The present invention also provides kits that contain a pharmaceutical formulation, together with instructions for the use of the formulation. Preferably, a commercial package will contain one or more unit doses of the formulation. Formulations which are light and/or air sensitive may require special packaging and/or formulation. For example, packaging may be used which is opaque to light, and/or sealed from contact with ambient air, and/or formulated with suitable coatings or excipients.

The formulations of the invention can be provided alone or in combination with other compounds (for example, small molecules, nucleic acid molecules, peptides, or peptide analogues), in the presence of a carrier or any pharmaceutically or biologically acceptable carrier. As used herein “pharmaceutically acceptable carrier” or “excipient” includes any and all solvents, dispersion media, coatings, antibacterial and antifungal agents, isotonic and absorption delaying agents, and the like that are physiologically compatible. The carrier can be suitable for any appropriate form of administration. Pharmaceutically acceptable carriers generally include sterile aqueous solutions or dispersions and sterile powders. Supplementary active compounds can also be incorporated into the formulations.

An “effective amount” of a formulation according to the invention includes a therapeutically effective amount or a prophylactically effective amount. A “therapeutically effective amount” refers to an amount effective, at dosages and for periods of time necessary, to achieve the desired therapeutic result. A therapeutically effective amount of a formulation may vary according to factors such as the disease state, age, sex, and weight of the individual, and the ability of the compound to elicit a desired response in the individual. Dosage regimens may be adjusted to provide the optimum therapeutic response. A therapeutically effective amount may also be one in which any toxic or detrimental effects of the formulation or active compound are outweighed by the therapeutically beneficial effects. A “prophylactically effective amount” refers to an amount effective, at dosages and for periods of time necessary, to achieve the desired prophylactic result. Typically, a prophylactic dose is used in subjects prior to or at an earlier stage of disease, so that a prophylactically effective amount may be less than a therapeutically effective amount. For any particular subject, the timing and dose of treatments may be adjusted over time (e.g., timing may be daily, every other day, weekly, monthly) according to the individual need and the professional judgment of the person administering or supervising the administration of the compositions.

A drug interaction is a therapeutic circumstance in which a substance affects the activity of a drug, i.e. the physiological effects of the drug are increased or decreased, or the substance and the drug together produce a new effect that neither produces on its own. In the context of an interaction between active pharmaceutical ingredients, this is known as a drug-drug interaction. Drug interactions occur on pharmacodynamic and pharmacokinetic levels, and may be positive or negative. Pharmacodynamic interactions are generally understood to be those in which drugs influence each other's effects directly. Pharmacokinetic interactions involve the reciprocal influences of disparate active ingredients on the absorption, distribution, metabolization, and/or elimination of each active ingredient. One category of positive drug interactions involves degrees of synergy between disparate active ingredients. Other positive drug interactions may include any therapeutically beneficial pharmacodynamic and/or pharmacokinetic interaction in which the therapeutic benefit of the combined use of the active ingredients, for example in a particular dosage regimen, is improved compared to the individual use of the active ingredients, for example in a comparable dosage regimen.

In therapeutic applications, synergy between active ingredients occurs when an observed combined therapeutic effect is greater than the sum of therapeutic effects of individual active ingredients, or a new therapeutic effect is produced that the active ingredients could not produce alone. Accordingly, when components of a formulation are present in synergistically effective amounts, the formulation yields a therapeutic effect that is greater than would be achieved by the individual active ingredients administered alone at comparable dosages. In this context, the enhancement of therapeutic effect may take the form of increased efficacy or potency and/or decreased adverse effects. The synergistic effect may be mediated in whole or in part by the pharmacokinetics and/or pharmacodynamics of the active ingredients in a subject, so that the amount and proportion of the ingredients in the formulation may be synergistic in vivo. This in vivo synergy may be effected with a formulation that includes the active ingredients in amounts and proportions that are also synergistic in in vitro assays of efficacy. As used herein, the term “synergistically effective amounts” accordingly refers to amounts that are synergistic in vivo and/or in vitro. A numeric quantification of synergy is often expressed as a fractional inhibitory concentration index (FICI), which represents the sum of the fractional inhibitory concentrations (FICs) of each drug tested, where the FIC is determined for each drug by dividing the minimum inhibitory concentration (MIC, the lowest concentration of the drug which prevents visible growth of the bacterium in a standard in vitro assay—standard colorometric assay based on resazurin) of each drug when used in combination by the MIC of each drug when used alone. In very general terms, a FICI lower or higher than 1 indicates positively correlated activity (at least additive or potentiation) or an absence of positive interactions, respectively. More definitively, synergy of two compounds may be conservatively defined as a FICI of ≤0.5 (see Odds, 2003); partial synergy or potentiation corresponding to a FICI of >0.5 to ≤0.75, no interaction (indifference) corresponding to a FICI of >1 to ≤4; and antagonism corresponding to a FICI of >>4. (as described and used by Joung D K et al. and Rakoliya K et al.)

### EXAMPLES

To illustrate the positive antibacterial interaction between silver and cannabinoids, the following examples include assays involving gram positive bacteria. Six types of cannabinoid were tested (CBC, CBD, CBG, CBCA, CBDA, and CBGA) in combination with silver nitrate (AgNO3) or silver nanoparticles (AgNP). In these examples, 20 mg/L of silver nanoparticles of −20 nm diameter, provided in 0.2 mM sodium citrate, or 1 mg/mL of silver nanoparticles of −10 nm diameter, provided in 2 mM sodium citrate, were used to prepare the silver nanoparticle treatments used. Methicillin-resistant Staphylococcus aureus (MRSA USA300) was used as an illustrative gram positive bacteria. Escherichia coli (E. coli strain K12) was used as an illustrative gram negative bacteria.

Antibacterial growth was measured using a checkerboard analysis or qualitatively using agar plates with zones of inhibition. Viability was measured by the number of colony forming units (CFU) over time.

Using checkerboard analysis, the fractional inhibitory concentration index (FICI) was calculated for the 96-well plate test to illustrate synergy. FICI indices were interpreted as follows: ≤0.5, synergy; <0.5-≤0.75 partial synergy or potentiation, 0.75-≤1.0, additive effect; indifference; and >1.0-≤4.0, antagonism as described and used by Joung D K et al and Rakholiya K et al.

In the context of the present disclosure, synergism occurs when two or more compounds interact in ways that mutually enhance, amplify or potentiate each other's effect more significantly than the simple sum of the effects of the compounds when used separately. Synergism accordingly contrasts with antagonism, in which a combination of compounds is antagonistic if their joint effect is weaker than the sum of effects of the individual agents or weaker than the effect of either individual agent. An additive interaction is the effect where the combined action is equivalent to the sum of the activities of each drug when used alone. An indifferent interaction between treatments occurs if their joint effect is equal to the effect of either of the individual agents, alone.

### General Methods:

### Bacterial Strains and Growth:

MRSA strain, USA300, was cultured in Luria-Bertani (LB) medium and inoculated at 37 C.

Escherichia coli (E. coli), strain K12, was cultured in Luria-Bertani (LB) medium and inoculated at 37 C.

### Checkerboard Assays:

Cannabinoids or silver (expressed as the concentration of silver not the salt) were serially diluted 2-fold across the 96-well plate (Costar, catalog no. 3370) followed by addition of 100 μL of bacterial cultures with an OD₆₀₀ of 0.0025. Cannabinoids concentrations ranged from 16 mg/L to 0.125 mg/L, silver nitrate ranged from 32 mg/L to 0.31 mg/L, silver nanoparticles ranged from 10 mg/L to 0.01 mg/L, and silver sulfate ranged from 10 mg/L to 0.01 mg/L. Plates were wrapped with aluminum foil and incubated for 24 hours. Wells turbidity were then analyzed using Varioskan™ microplate reader.

### FICI Computation:

FICI was calculated in a checkerboard assay based on the turbidity of the wells. FIC of each agent was determined as the ratio of the minimal inhibitory concentration MIC of one agent in the presence of the other agent to the MIC of that agent alone. FICI was consequently computed as the sum of each agent's FIC. Note: FIC indices (FICI) were interpreted as follows: ≤0.5, synergy; <0.5-≤0.75 partial synergy, 0.75-1.0, additive effect; indifference; and >4.0, antagonism as described by Joung D K et al.

### Kill-Curve Test:

For each test tube, 1 mL of culture at OD₆₀₀ of 0.005 was added to 1 mL of LB medium containing antibiotics to reach target sub-MIC concentrations of each compound. Samples of 100 μL was extracted from each tube at the determined time stamps followed by 10-fold serial dilutions. 10 μL of each dilution was then added on the LB agar plates that were subsequently incubated for 24 hours. Colonies were inspected and results quantified as log CFU/mL.

### Example 1: Checkerboard Analysis of the Effect of Cannabinoids and Silver Nitrate Combinations on MRSA Growth

TABLE A
Fractional Inhibitory Concentration Indices (FICI) of Silver
Nitrate in combination with Cannabinoids in MRSA (USA300)

| | FICI |
|---|---|
| CBD | 2 |
| CBDA | 2 |
| CBC | 0.375 |
| CBCA | 2 |
| CBG | 0.625 |
| CBGA | 0.53 |

As shown in Table A, CBC and silver nitrate were strongly synergistic in combination with a low FICI score of 0.375. CBGA combined with silver nitrate gave a FICI score of 0.53 just above the synergy descriptor but at the high end of partial synergy. With a FICI score of 0.625, CBG is partially synergistic against MRSA when combined with silver nitrate. There was no improved antibiotic affect against MRSA using CBD, CBDA or CBCA in combination with silver nitrate and FICI scores were all 2.

TABLE B
Fractional Inhibitory Concentration Indices (FICI) of Silver Nanoparticles
in combination with Cannabinoids in MRSA (USA300)

| | FICI |
|---|---|
| CBC | 0.141 |
| CBGA | 0.375 |
| CBG | 0.625 |

As shown in Table B, CBC and CBGA were each synergistic against MRSA in combination with silver nanoparticles with FICI scores of 0.141 and 0.375, respectively. With a FICI score of 0.625, partial synergy was found between CBG and silver nanoparticles.

### Example 2: Kill Curve Analyses of CBG with Silver Nitrate Against MRSA

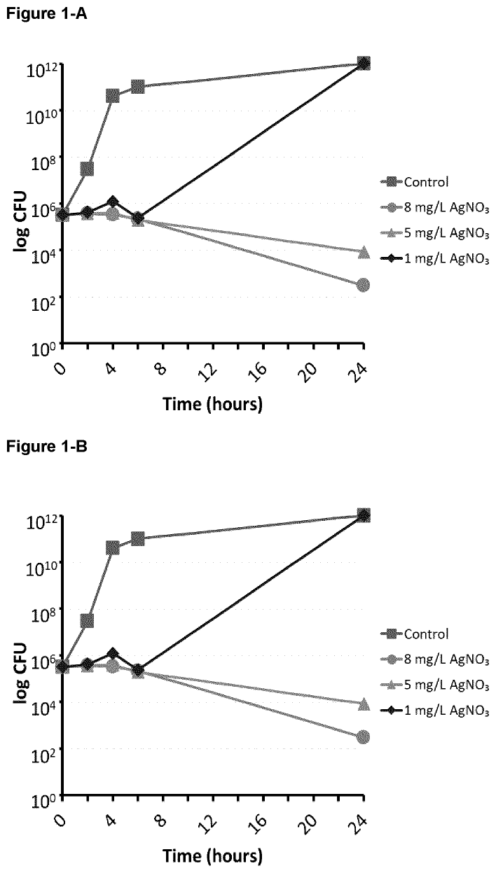

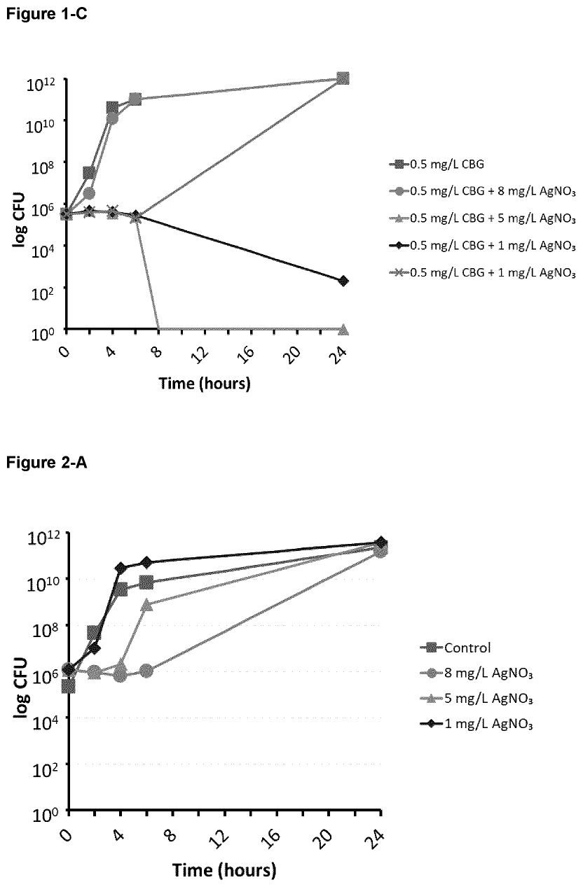

Using MRSA (strain USA300) CBG was found to have an MIC of 2 mg/L. Silver as silver nitrate was found to have an MIC of 16 mg/L, although there was a time dependency of the efficacy of silver nitrate. Using a concentration of silver nitrate at 1 mg/L, bacterial growth was inhibited for 6 hours but by 24 hours full bacterial growth had occurred. Following treatment with 5 and 8 mg/L silver nitrate, bacterial growth was inhibited at 24 hours (FIG. 1-A). However, the addition of ½×MIC CBG (1 mg/L) to silver nitrate 5 and 8 mg/L not only inhibited bacterial growth, the combination gave a fully bactericidal effect (i.e., elimination of detectable CFU) that was rapid, occurring as early as 2 hours following treatment (FIG. 1-B). Furthermore, the bactericidal effect remained 24 hours after treatment. Even the addition of ½×MIC CBG to 1 mg/L silver nitrate gave a bactericidal effect 2 hours following treatment and the effect remained for up to 6 hours. A similar effect occurred using CBG at just ¼×MIC (0.5 mg/L) whereby the combination with silver nitrate at 5 and 8 mg/L was perceptibly bactericidal (FIG. 1-C).

These data demonstrate a positive drug-drug interaction with a silver-containing medicament used in combination with a cannabinoid, in particular illustrating a stronger antibiotic action of using CBG in combination with silver nitrate as opposed to using either compound on its own.

### Example 3. Kill Curve Analyses of CBC with Silver Nitrate Against MRSA

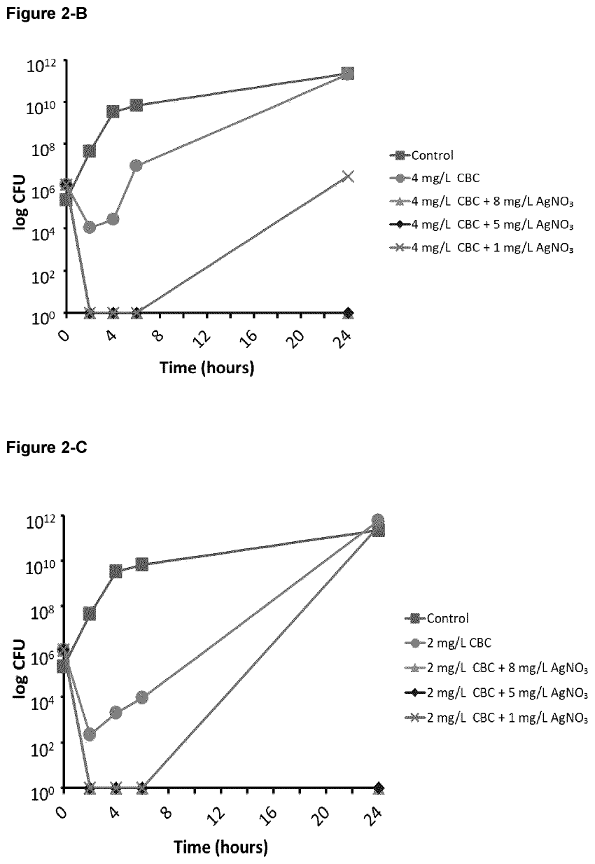

Using MRSA (strain USA300) CBC was found to have an MIC of 8 mg/L. Silver as silver nitrate was found to have an MIC of 16 mg/L. Using a concentration of silver nitrate at 8 mg/L, bacterial growth was inhibited for 6 hours and with 5 mg/L it was inhibited for 4 hours and at 1 mg/L there was no inhibition (FIG. 2-A). At all concentrations of silver nitrate used, full bacterial growth occurred at 24 hours. Using ½×MIC CBC (4 mg/L) alone, bacterial growth was inhibited for 6 hours but by 24 hours full bacterial growth had occurred (FIG. 2-B). However, when ½×MIC CBC was used in combination with silver nitrate 8 and 5 mg/L the combinations not only inhibited bacterial growth, they were fully bactericidal, eliminating any detectable CFU from 2 through 24 hours following treatment (FIG. 2-B). Even ½×MIC CBC with silver nitrate 1 mg/L gave a complete bactericidal effect (eliminated detectable CFU) 2 hours following treatment and the effect persisted for 6 hours. At 24 hours there remained a strong inhibition of bacterial growth (FIG. 2-B).

Almost identical results were obtained using ¼×MIC CBC (2 mg/L) with silver nitrate (FIG. 2-C) with the exception that the combination with silver nitrate at 1 mg/L gave strong inhibition for 6 hours but this did not last for 24 hours as was the case with ½×MIC CBC.

These data demonstrate a positive drug-drug interaction with a silver-containing medicament used in combination with a cannabinoid, in particular illustrating a stronger antibiotic effect when using CBC in combination with silver nitrate in MRSA bacteria, compared to the antibiotic effect of using either compound on its own.

### Example 4. Kill Curve Analyses of CBC with Silver Nanoparticles Against MRSA

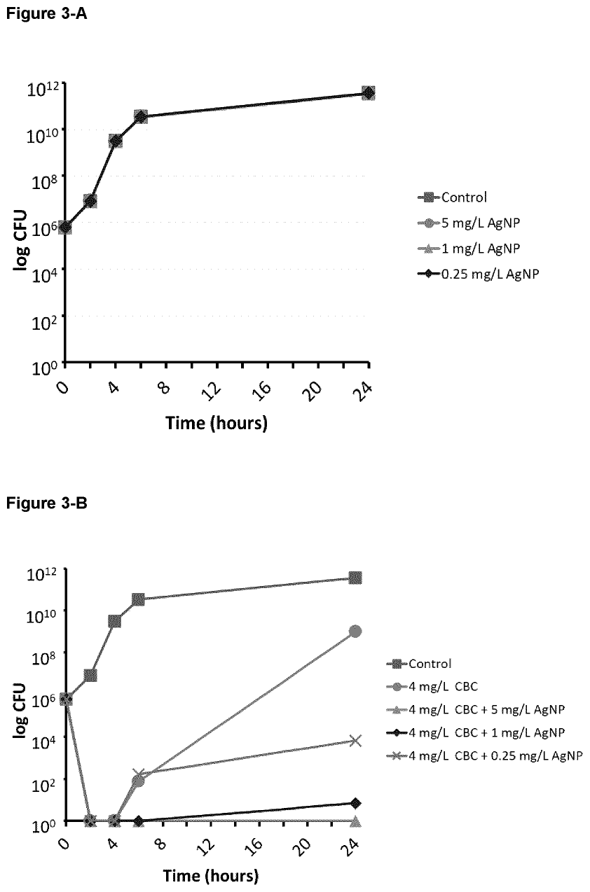

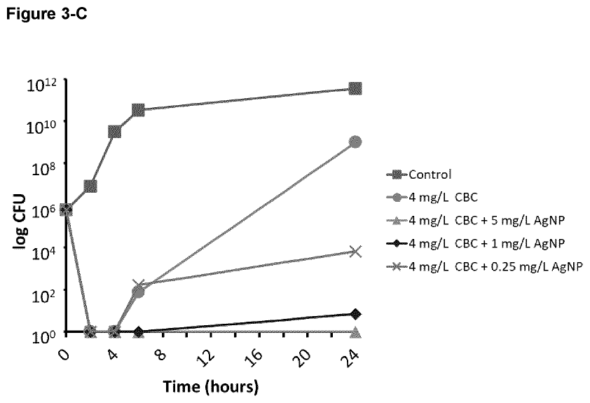

Using MRSA (strain USA 300), CBC was found to have an MIC of 8 mg/L. Silver as silver nanoparticles (AgNP) had an MIC of 40 mg/L. In a kill curve over time, AgNP did not show inhibition of MRSA at any of the sub-MIC concentrations (⅛, ⅕ and 1/40 MIC) tested (FIG. 3-A). Using CBC at ½×MIC (4 mg/L), there was inhibition of MRSA growth seen at 2, 4 and 6 hours, however, by 24 hours, MRSA growth returned to levels approximating that of 0×MIC treatment (FIG. 3-B). With the addition of silver nanoparticles (⅛×MIC or 5 mg/L) to ½×MIC CBC, there was a full bactericidal effect (complete elimination of CFU) as early as 2 hours following treatment which persisted through 24 hours. ½×MIC CBC with 1/40×MIC AgNP (1 mg/L) gave a nearly identical full bactericidal effect which was rapid (within 2 hours) and persisting for 24 hours. Surprisingly, ½×MIC CBC with 1/160×MIC AgNP (0.25 mg/L) produced a strong bactericidal effect which was far greater than that seen with ½×MIC CBC alone.

Similar results were obtained using ¼×MIC CBC (2 mg/L, FIG. 3-C), except all combined inhibitory effects with the addition of AgNPs were weaker and less durable as compared to combinations involving ½×MIC CBC.

These data demonstrate a positive drug-drug interaction with silver nanoparticles used in combination with a cannabinoid. This example illustrates a far stronger antibiotic action of CBC in combination with silver nanoparticles, compared to the antibiotic effect of either compound on its own. The observed antibiotic effect of the two compounds in combination also exceeds the additive effect one may expect when combining the two compounds, considering that silver nanoparticles alone gave a null antibiotic effect at the concentrations tested.

### Example 5. Agar Plate Test of CBGA with Silver Nanoparticles on MRSA Growth

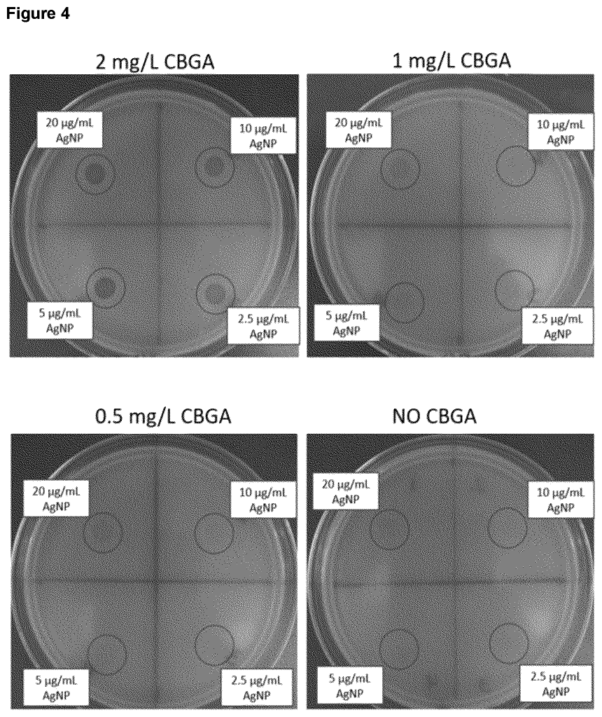

Four LB agar plates were prepared with MRSA culture at OD₆₀₀ of 0.005. (FIG. 4) Each plate contained different concentrations of CBGA (no CBGA, ½ MIC, ¼ MIC, and ⅛ MIC). All plates were then divided into quadrants, each of which received 8 μL of AgNP at different concentrations from 20 mg/L serially diluted by 2-fold down to 2.5 mg/L in 0.2 mM sodium citrate.

Without any CBGA in the agar the addition of 8 μL of silver nanoparticles at 2.5, 5, 10 or 20 mg/L had no effect on bacterial growth (lower right caption). However, when CBGA at ⅛th MIC (lower left caption) or ¼ MIC was incorporated into the agar, there was a concentration dependent darkening in the photo (antibiotic combination-mediated inhibition of bacterial growth allowed visualization of black background). This can best be visualized in the top left quadrant of both captions for the 20 mg/L silver nanoparticle sample where a clear dark circle can be seen with a less dense dark circle seen at 10 mg/L (top right quadrant of each plate). Using ½ the MIC in the agar resulted in strong inhibition of MRSA growth in all wells (top left caption). The addition of 20 mg/L (top left quadrant) of silver nanoparticles resulted in the strongest darkness and strongest antibiotic effect but it was difficult to distinguish densities in this plate due to the strong antibiotic effect of CBGA at ½ MIC.

These results demonstrate that the antibiotic effect of CBGA is augmented strongly in a concentration dependent manner by the addition of silver nanoparticles.

### Example 6. Antibiotic Effects of Polymer Films or Polymer Coated Urethral Catheters Using Combinations of CBC, CBG and CBGA with Either Silver Nitrate or Silver Nanoparticles

PVA films were made by solvent casting using 33 μl drops of 2.5% PVA (88% hydrolyzed, 125 KDa molecular weight) mixed with CBC, CBG or CBGA (2% w/w to PVA). In some samples, silver nitrate or silver nanoparticles alone were added at 2% Silver to PVA (w/w) with or without the cannabinoid. 3 mm sections of a urethral catheter (BARDEX® BARD®) were cut and coated with the same 33 μl volume of PVA/Silver/cannabinoid used to make films. Films and coated catheter sections were dried overnight in the dark. Five cavities were created in the LB agar plates containing MRSA (strain USA 300) at OD₆₀₀ of 0.005. PVA films and catheters coated with PVA films were then placed in the cavities and moistened with 20 μL of deionized distilled water. Plates were incubated for 24 hours at 37° C.

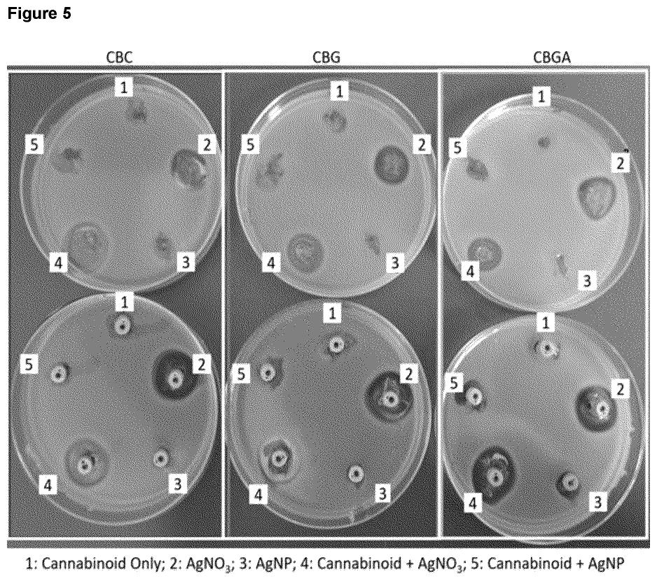

The upper plates show the films and the lower plates the coated catheter parts (FIG. 5). Inhibition of bacterial growth is seen as a darkish ring against the opaque MRSA background. All films and coated catheter sections for CBC, CBG and CBGA as well as silver nitrate and silver nanoparticles showed some level of inhibition of bacterial growth. This inhibition was relatively minor for the cannabinoid alone (position 1 in all plates) and for silver nanoparticles alone. Silver nitrate alone shows a strong inhibition of bacterial growth in all plates. This very strong inhibition masks the assessment of an increase in antibacterial action when combined with cannabinoid, although there was evidence of an increased antibiotic effect for CBGA combined with silver nitrate (compare positions 2 and 4).

For silver nanoparticles there was an increased antibiotic effect (larger area or darkness of ring) when combined with CBC, CBG or CBGA for both films (upper plates) or coated catheter sections (lower plates), as can be visualized comparing positions 3 and 5 in all six plates.

This example illustrates a temporal dosing effect, related to the staged release of cannabinoid and silver-containing medicaments from the PVA. The PVA swells to form a hydrogel (a property that is particularly beneficial in wound healing applications or in a catheter coating), the swollen hydrogel then releases the antibiotic agents over time in a staged sequence. Silver nitrate is very soluble, and as a result is released relatively quickly, creating relatively high local concentrations of silver, whereas silver nanoparticles and cannabinoids (largely insoluble) are released very slowly, maintaining effective combined antibiotic efficacy over time. As exemplified, all combinations of silver nitrate or silver nanoparticles with all 3 cannabinoids (CBC, CBG and CBGA) inhibit MRSA growth. Furthermore, there is evidence of an increased antibiotic effect for silver nitrate with CBGA and for silver nanoparticles for all three cannabinoids. These effects are demonstrably consistent in alternative impregnated matrices—the PVA films and the coated catheters.

### Example 7. Kill Curve Analyses of CBGA with Silver Nanoparticles Against MRSA

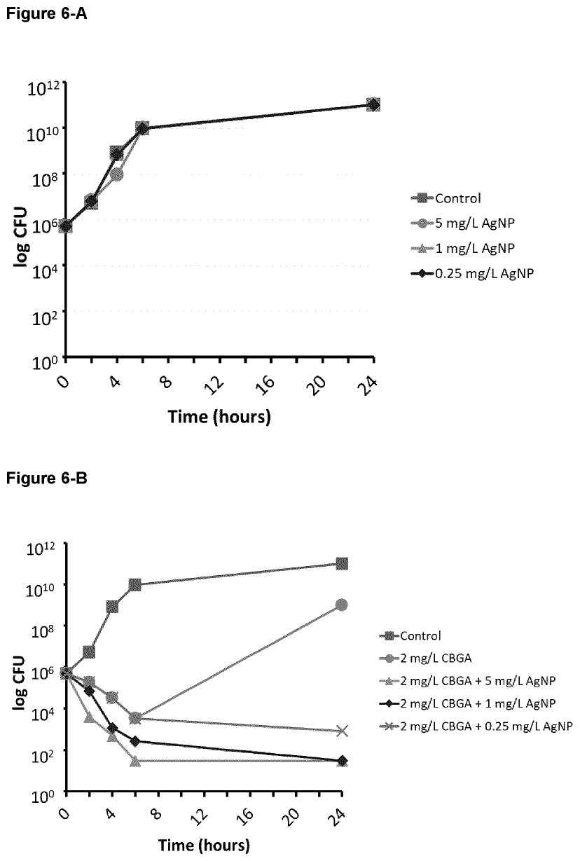

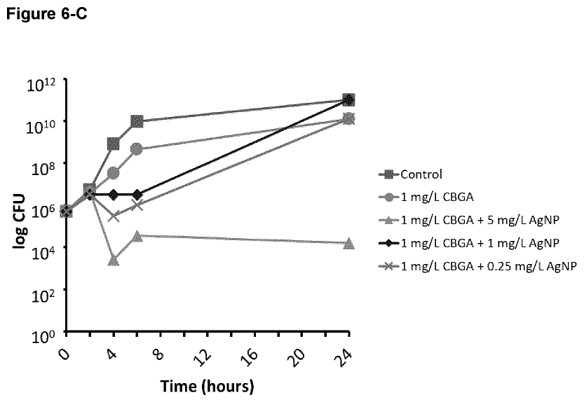

Using MRSA (strain USA 300), CBGA was found to have an MIC of 4 mg/L. Silver as silver nanoparticles (AgNP) had an MIC of 40 mg/L. In a kill curve analysis over time, AgNP did not show inhibition of MRSA at any of the concentrations tested (FIG. 6-A). Using CBGA at ½×MIC (2 mg/L), there was inhibition of MRSA growth seen at 4 and 6 hours, however, by 24 hours, MRSA growth returned to levels approximating that of 0×MIC treatment (FIG. 6-B). With the addition of silver nanoparticles (⅛×MIC or 5 mg/L) to ½×MIC CBGA, there was a full bactericidal effect (complete elimination of CFU) following treatment which persisted through 24 hours. ½×MIC CBGA with 1/40×MIC AgNP (1 mg/L) gave a nearly identical full bactericidal effect which was rapid and persisted for 24 hours. Surprisingly, ½×MIC CBGA with 1/160×MIC AgNP (0.25 mg/L) produced a strong bactericidal effect which was far greater than that seen with ½×MIC CBGA alone.

Similar results were obtained using ¼×MIC CBGA (1 mg/L, FIG. 6-C), except all combined inhibitory effects with the addition of AgNPs were weaker and less durable as compared to combinations involving ½×MIC CBGA.

These data demonstrate a positive drug-drug interaction with silver nanoparticles used in combination with a cannabinoid. This example illustrates a far stronger antibiotic action of CBGA in combination with silver nanoparticles, compared to the antibiotic effect of either compound on its own. The observed antibiotic effect of the two compounds in combination also exceeds the additive effect one may expect when combining the two compounds, considering that silver nanoparticles alone gave a null antibiotic effect at the concentrations tested.

### Example 8. Kill Curve Analyses of CBG with Silver Nanoparticles Against MRSA

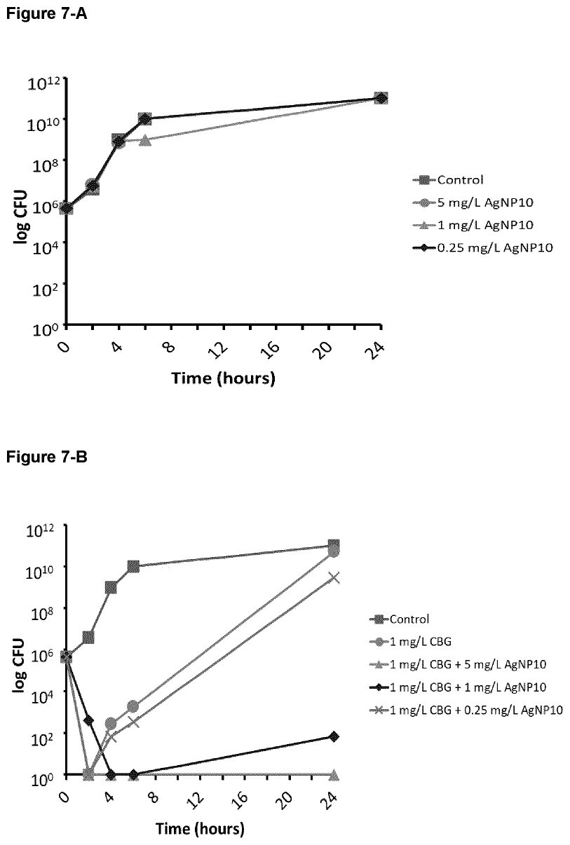

Using MRSA (strain USA 300), CBG was found to have an MIC of 2 mg/L. Silver as silver nanoparticles (AgNP) had an MIC of 40 mg/L. In a kill curve analysis over time, AgNP did not show any inhibition of MRSA at the concentrations tested (FIG. 7-A). Using CBG at ½×MIC (1 mg/L), inhibition of MRSA growth was seen at 2, 4 and 6 hours, however, by 24 hours, MRSA growth returned to levels equal to that of 0×MIC treatment (FIG. 7-B). With the addition of silver nanoparticles (⅛×MIC or 5 mg/L) to ½×MIC CBG, there was a rapid, full bactericidal effect (complete elimination of CFU) 2 hours following treatment which persisted through 24 hours. ½×MIC CBG with 1/40×MIC AgNP (1 mg/L) gave a highly comparable bactericidal effect which was rapid and persisted for 24 hours. Surprisingly, ½×MIC CBG with 1/160×MIC AgNP (0.25 mg/L) also produced a bactericidal effect which was greater than that seen with ½×MIC CBG alone.

These data demonstrate a positive drug-drug interaction with silver nanoparticles used in combination with a cannabinoid. This example illustrates a stronger antibiotic action of CBG in combination with silver nanoparticles compared to the antibiotic effect of either compound on its own. The observed antibiotic effect of the two compounds in combination also exceeds the additive effect one may expect when combining the two compounds, considering that silver nanoparticles alone gave a null antibiotic effect at the concentrations tested.

### Example 9. Kill Curve Analyses of CBD with Silver Nanoparticles Against MRSA

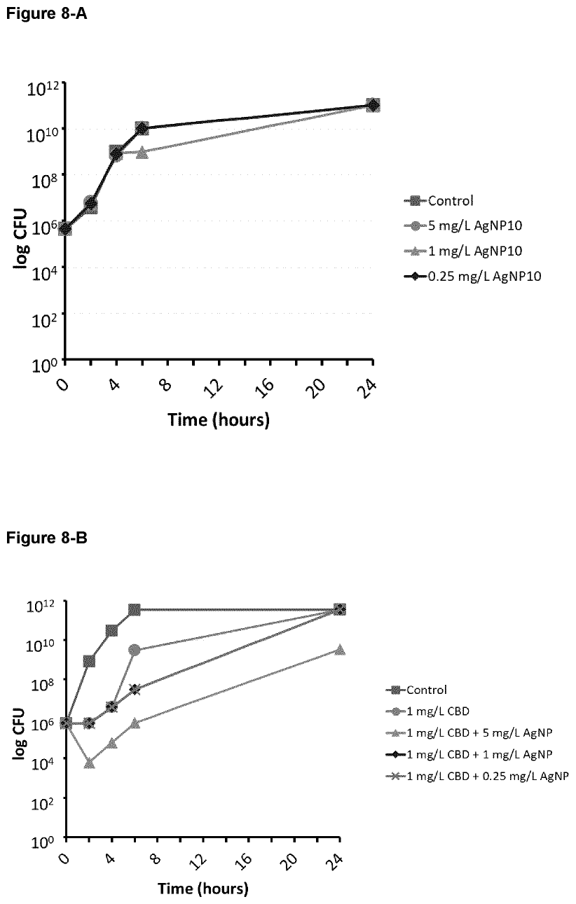

Using MRSA (strain USA 300), CBD was found to have an MIC of 2 mg/L. Silver as silver nanoparticles (AgNP) had an MIC of 40 mg/L. In a kill curve analysis over time, AgNP did not show any inhibition of MRSA at the concentrations tested (FIG. 8-A). Using CBD at ½×MIC (1 mg/L), inhibition of MRSA growth was seen at 2, 4 and 6 hours, however, by 24 hours, MRSA growth returned to levels equal to that of 0×MIC treatment (FIG. 8-B). With the addition of silver nanoparticles (⅛×MIC or 5 mg/L) to ½×MIC CBD, there was a rapid bactericidal effect 2 hours following treatment and a substantive decrease in MRSA growth over 24 hours compared to CBD alone.

These data demonstrate a positive drug-drug interaction with silver nanoparticles used in combination with a cannabinoid. This example illustrates a stronger antibiotic action of CBD in combination with silver nanoparticles compared to the antibiotic effect of either compound on its own. The observed antibiotic effect of the two compounds in combination also exceeds the additive effect one may expect when combining the two compounds, considering that silver nanoparticles alone gave a null antibiotic effect at the concentrations tested.

### Example 10. Kill Curve Analyses of CBCA with Silver Nanoparticles Against MRSA

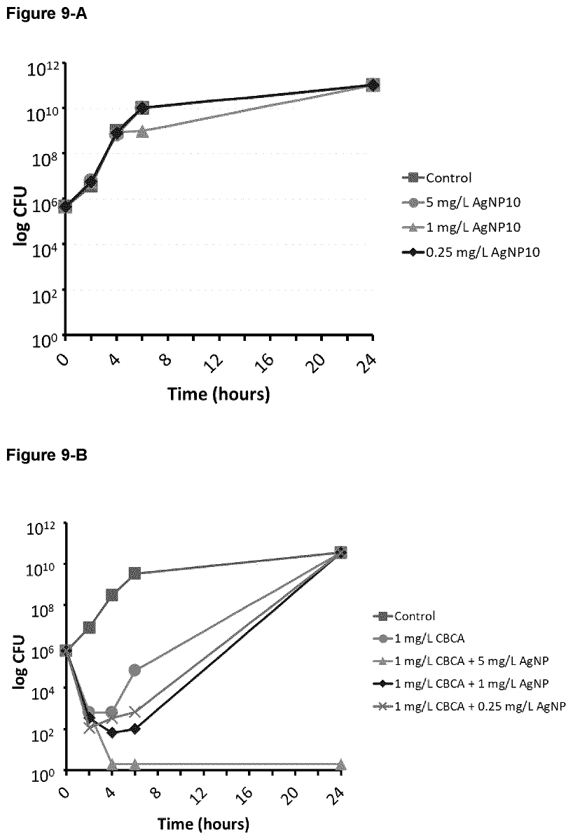

Using MRSA (strain USA 300), CBCA was found to have an MIC of 2 mg/L. Silver as silver nanoparticles (AgNP) had an MIC of 40 mg/L. In a kill curve analysis over time, AgNP did not show any inhibition of MRSA at the concentrations tested (FIG. 9-A). Using CBCA at ½×MIC (1 mg/L), inhibition of MRSA growth was seen at 2, 4 and 6 hours, however, by 24 hours, MRSA growth returned to levels equal to that of 0×MIC treatment (FIG. 9-B). With the addition of silver nanoparticles (⅛×MIC or 5 mg/L) to ½×MIC CBCA, there was a rapid bactericidal effect 2 hours following treatment and full elimination of CFU at 4 hours which persisted through 24 hours. Additionally, the addition of 1/40×MIC AgNP (1 mg/L) to ½×MIC CBCA resulted in a greater bactericidal effect against MRSA that that seen with CBCA alone.

These data demonstrate a positive drug-drug interaction with silver nanoparticles used in combination with a cannabinoid. This example illustrates a stronger antibiotic action of CBCA in combination with silver nanoparticles compared to the antibiotic effect of either compound on its own. The observed antibiotic effect of the two compounds in combination also exceeds the additive effect one may expect when combining the two compounds, considering that silver nanoparticles alone gave a null antibiotic effect at the concentrations tested.

### Example 11: Checkerboard Analysis of the Effect of Cannabinoids and Silver Combinations on E. coli Growth

TABLE C
Fractional Inhibitory Concentration Indices (FICI)
of Silver Sulfate and Silver Nanoparticles in combination
with Cannabinoids in E. coli.

| | FICI Silver Sulfate | FICI Silver Nanoparticles |
|---|---|---|
| CBD | 1 | 1.25 |
| CBDA | 0.52 | 0.73 |
| CBC | 0.63 | 0.73 |
| CBCA | 0.50 | 1.25 |
| CBG | 1 | 1.25 |
| CBGA | 0.75 | 0.73 |

As shown in Table C, the interaction between silver sulfate and CBCA was synergistic (FICI=0.50), while potentiation was observed with silver sulfate in combination with each of CBDA, CBC and CBGA. Such interactions were not observed between silver sulfate and CBD nor between silver sulfate and CBG. Checkerboard analyses using silver nanoparticles failed to produce any synergistic interaction with the cannabinoids tested, however, potentiation was observed with silver nanoparticles in combination with each of CBDA, CBC and CBGA.

### Example 12. Kill Curve Analyses of Cannabinoids with Silver Sulfate Against E. Coli

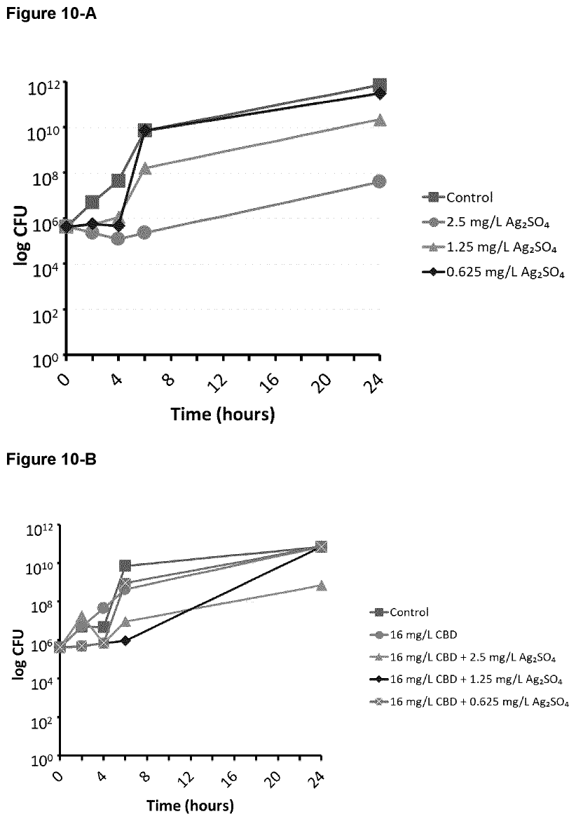

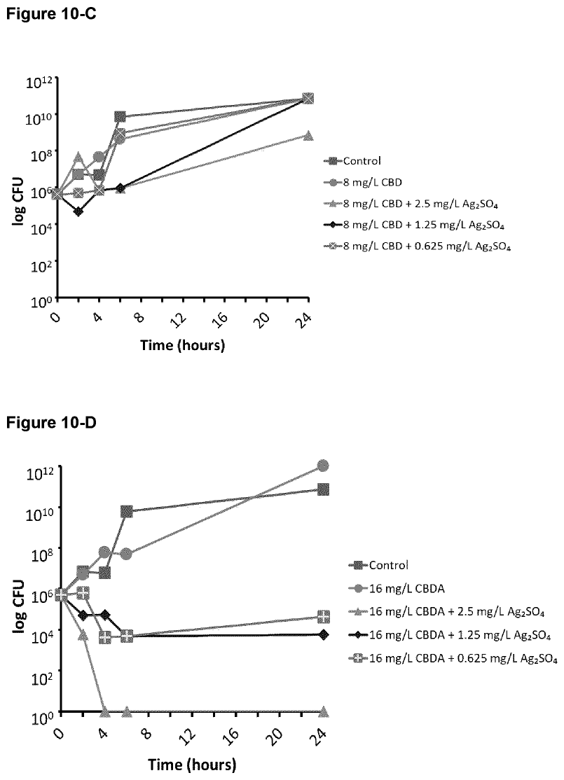

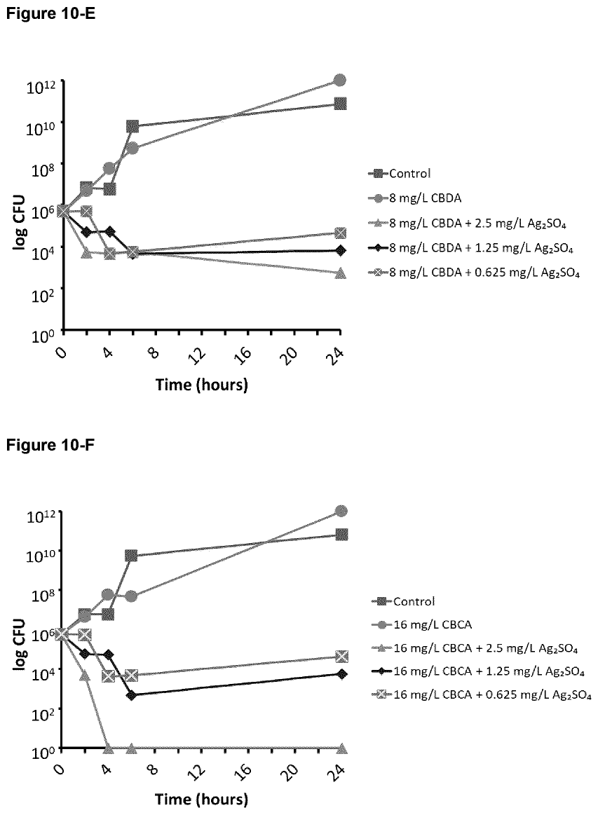

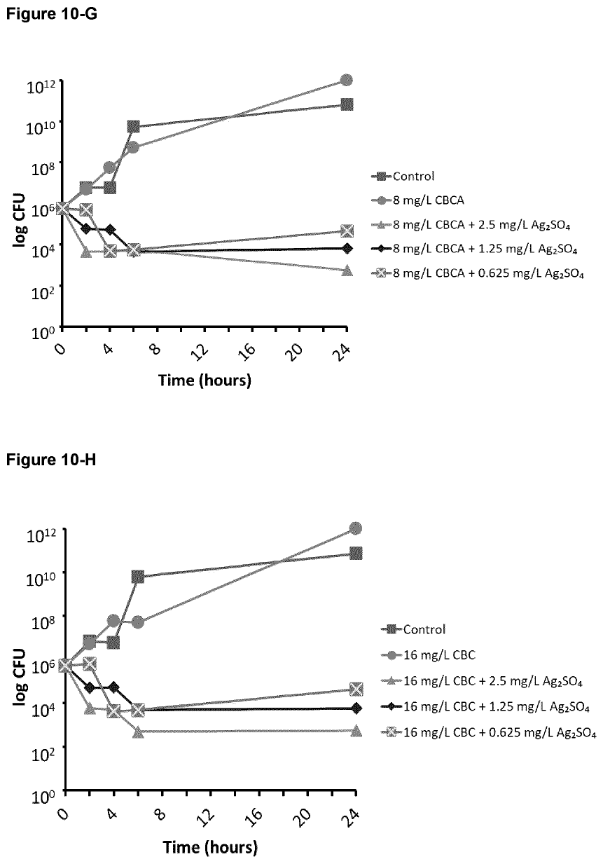

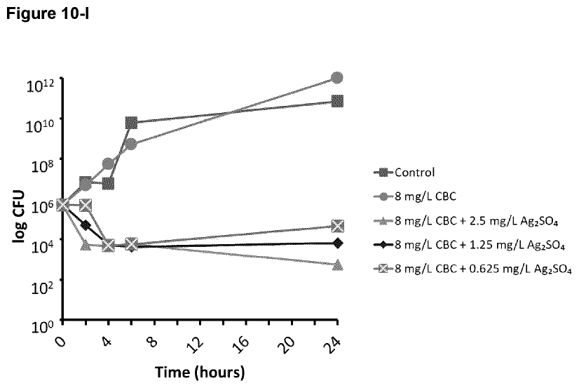

Against E. coli (strain K12), silver sulfate (AgSO₄) was found to have an MIC of 2.5 mg/L. In a kill curve analysis over time, treatment with 2.5 mg/L AgSO₄ showed inhibition of E. coli growth over a 6 hour period, no bactericidal effects (i.e., no net reduction in bacterial CFU) and approximately 1-log total increase in bacterial CFU at 24 hours (FIG. 10-A). In comparison, the control (no treatment) group had a 6-log increase in bacterial CFU at 24 hours. Treatment with 1.25 mg/L and 0.625 mg/L AgSO₄ showed some initial growth inhibition; however, at 4 hours, bacterial growth increased substantially and the growth curve over time resembled the control (no treatment) group (FIG. 10-A). As illustrated in FIG. 10-B, when treated with CBD alone at a concentration of 16 mg/L, no inhibition of E. coli growth was observed over 24 hours and the growth curve resembled that of the control (no treatment) group. The combination of 2.5 mg/L AgSO₄ and 16 mg/L CBD did not result in a substantial inhibition of E. coli growth over a 24 hour period (FIG. 10-B). The result of the combination resembled that of treatment with 2.5 mg/L AgSO₄ alone, shown in FIG. 10-A. Similar results were observed when 2.5 mg/L AgSO₄ was combined with 8 mg/L CBD (FIG. 10-C). In the case of CBDA treatment as shown in FIG. 10-D, treatment with 16 mg/L CBDA alone failed to achieve any inhibition of E. coli growth over 24 hours and the kill curve resembled that of the control (no treatment) group. Surprisingly, the addition of 2.5 mg/L AgSO₄ to 16 mg/L CBDA resulted in a substantial 6-log (99.9999%) reduction in E. coli CFU within 4 hours. This rapid bactericidal effect persisted through 24 hours (FIG. 10-D). Additionally, treatment with sub-MIC AgSO₄ concentrations of 1.25 mg/L and 0.625 mg/L in combination with 16 mg/L CBDA resulted in 2-log (99%) reductions in E. coli CFU and inhibition of bacterial growth through 24 hours (FIG. 10-D). As shown in FIG. 10-E, treatment with 8 mg/L CBDA alone failed to achieve any inhibitory effect on E. coli growth over 24 hours and the kill curve resembled that of the control (no treatment) group. When combined with 8 mg/L CBDA, 2.5, 1.25 and 0.625 mg/L AgSO₄ each showed 2-log (99%) reductions in E. coli CFU within 6 hours of treatment (FIG. 10-E). Each combination inhibited E. coli growth through 24 hours, with 2.5 mg/L AgSO₄ having the strongest inhibitory effect.

The anti-microbial effects observed from the combination of AgSO₄ with CBDA are therefore far in excess of additive effects, given the weak or non-existent anti-microbial effects of each compound on its own at the concentrations tested.

As shown in FIG. 10-F, treatment with 16 mg/L CBCA alone failed to achieve any inhibition of E. coli growth over 24 hours and the kill curve resembles that of the control (no treatment) group. Surprisingly, the addition of 2.5 mg/L AgSO₄ to 16 mg/L CBCA resulted in a substantial 6-log (99.9999%) reduction in E. coli CFU within 4 hours. This rapid bactericidal effect persisted through 24 hours (FIG. 10-F). Additionally, treatment with 1.25 mg/L AgSO₄ (sub-MIC concentration) in combination with 16 mg/L CBCA resulted in a 3-log (99.9%) reduction in E. coli CFU and inhibition of bacterial growth through 24 hours (FIG. 10-F). Furthermore, treatment with 0.625 mg/L AgSO₄ (sub-MIC concentration) in combination with 16 mg/L CBCA resulted in a 2-log (99%) reduction in E. coli CFU and inhibition of bacterial growth through 24 hours (FIG. 10-F). As shown in FIG. 10-G, treatment with 8 mg/L CBCA alone failed to achieve any inhibitory effect on E. coli growth over 24 hours and the kill curve resembles that of the control (no treatment) group. When combined with 8 mg/L CBCA, 2.5, 1.25 and 0.625 mg/L AgSO₄ each showed 2-log (99%) reductions in E. coli CFU within 6 hours of treatment (FIG. 10-G). Each combination inhibited E. coli growth through 24 hours, with 2.5 mg/L AgSO₄ having the strongest inhibitory effect.

The anti-microbial effects observed from the combination of AgSO₄ with CBCA are therefore far in excess of additive effects, given the weak or non-existent anti-microbial effects of each compound on its own at the concentrations tested.

As shown in FIG. 10-H, treatment with 16 mg/L CBC alone failed to achieve any inhibition of E. coli growth over 24 hours and the kill curve resembles that of the control (no treatment) group. Surprisingly, the addition of 2.5 mg/L AgSO₄ to 16 mg/L CBC resulted in a 3-log (99.9%) reduction in E. coli CFU within 6 hours. This rapid anti-microbial effect persisted through 24 hours (FIG. 10-H). Additionally, treatment with 1.25 mg/L and 0.625 mg/L AgSO₄ (sub-MIC concentrations) in combination with 16 mg/L CBC resulted in 2-log (99%) reductions in E. coli CFU within 6 hours and continuous inhibition of bacterial growth through 24 hours (FIG. 10-H). As shown in FIG. 10-I, treatment with 8 mg/L CBC alone failed to achieve any inhibitory effect on E. coli growth over 24 hours and the kill curve resembles that of the control (no treatment) group. When combined with 8 mg/L CBC, 2.5, 1.25 and 0.625 mg/L AgSO₄ each showed 2-log (99%) reductions in E. coli CFU within 4 hours of treatment (FIG. 10-I). Each combination inhibited E. coli growth through 24 hours, with 2.5 mg/L AgSO₄ having the strongest inhibitory effect.

The anti-microbial effects observed from the combination of AgSO₄ with CBC are therefore far in excess of additive effects, given the weak or non-existent anti-microbial effects of each compound on its own at the concentrations tested.

In summary, this example illustrates that silver sulfate has a weak inhibitory effect on E. coli growth when administered alone at 2.5 mg/L, and very little inhibitory effects are seen at lower silver sulfate concentrations. Furthermore, cannabinoids CBD, CBDA, CBCA and CBC administered alone fail to achieve any detectable inhibition of E. coli growth, at concentrations up to 16 mg/L. That select cannabinoid combinations with silver sulfate result in rapid bactericidal activity against E. coli at the concentrations tested is surprising and unexpected, particularly given that the effect is only seen in the case of specific cannabinoids (i.e., no increase in antimicrobial activity is seen when silver sulfate is combined with CBD, whereas 99.9999% killing of E. coli within 4 hours is observed when silver sulfate is combined with CBDA or CBCA).

### REFERENCES

Abdelaziz, A., (1982) Studies on the antimicrobial activity of cannabinoids. MS thesis, Ohio State University.
André, C. M.; Hausman, J.-F.; Guerriero, G. (2016). “Cannabis sativa: The Plant of the Thousand and One Molecules”. Frontiers in Plant Science. 7: 19.
Appendino, G., G. Chianese & 0. Taglialatela-Scafati, (2011) Cannabinoids: occurrence and medicinal chemistry. Curr Med Chem 18: 10854099.
Appendino, G., Gibbons, S., Giana, A., Pagani, A., Grassi, G., Stavri, M., Smith, E., and Rahman, M. M., 2008, “Antibacterial Cannabinoids from Cannabis Sativa: A Structure-Activity Study,” J. Nat. Prod., 71(8), pp. 1427-1430.
Appendino, G., S. Gibbons, A. Giana, A. Pagani, G. Grassi, M. Stavri, E. Smith & M. M. Rahman, (2008) Antibacterial cannabinoids from Cannabis sativa: a structure-activity study. J Nat Prod 71: 1427-1430.
Arias, C. A. & B. E. Murray, (2012) The rise of the Enterococcus: beyond vancomycin resistance. Nat Rev Microbiol 10: 266-278.
Arias, C. A., D. Panesso, D. M. McGrath, X. Qin, M. F. Mojica, C. Miller, L. Diaz, T. T. Tran, S. Rincon, E. M.
Arthur, M. & P. Courvalin, (1993) Genetics and mechanisms of glycopeptide resistance in enterococci. Antimicrob Agents Chemother 37: 1563-1571.
Ashurst, P. R., Bohlmann, F., Farkas, L., Gaoni, Y., KLING, H., Mechoulam, R., Morrison, G. A., Pallos, L., Romo, J., Romo De Vivar, A., Sutherland, J. K., and Waldschmidt-Leitz, E., 1967, Progress in the Chemistry of Organic Natural Products, Springer Science & Business Media.
Baltz R H (December 2006). “Molecular engineering approaches to peptide, polyketide and other antibiotics”. Nature Biotechnology. 24 (12): 1533-40.
Barbu, J. Reyes, J. H. Roh, E. Lobos, E. Sodergren, R. Pasqualini, W. Arap, J. P. Quinn, Y. Shamoo, B. E. Murray & G. M. Weinstock, (2011) Genetic basis for in vivo daptomycin resistance in enterococci. N Engl.' Med 365: 892-900.
Barnea, Y., Weiss, J., and Gur, E., 2010, “A Review of the Applications of the Hydrofiber Dressing with Silver (Aquacel Ag®) in Wound Care,” Ther. Clin. Risk Manag., 6(1), pp. 21-27.
Bell, A., 2005, “Antimalarial Drug Synergism and Antagonism: Mechanistic and Clinical Significance,” FEMS Microbiol. Lett., 253(2), pp. 171-184.
Berenbaum, M. C. 1978. A method for testing for synergy with any number of agents. J. Infect. Dis. 137:122-130.
Bergin, S., and Wraight, P., 2006, “Silver based wound dressings and topical agents for treating diabetic foot ulcers,” Cochrane Database of Systematic Reviews, (1).
Chakraborty, S., Afaq, N., Singh, N., and Majumdar, S., 2018, “Antimicrobial Activity of Cannabis Sativa, Thuja Orientelis and Psidium Guajava Leaf Extracts against Methicillin-Resistant Staphylococcus Aureus,” J. Integr. Med., 16(5), pp. 350-357.
Chopra, I., 2007, “The increased use of silver-based products as antimicrobial agents: a useful development or a cause for concern?” Journal of Antimicrobial Chemotherapy, 59, pp. 587-590.
Consroe, P., J. Laguna, J. Allender, S. Snider, L. Stern, R. Sandyk, K. Kennedy & K. Schram, (1991) Controlled clinical trial of cannabidiol in Huntington's disease. Pharmacol Biochem Behav 40: 701-708.
Cunha, J. M., E. A. Carlini, A. E. Pereira, O. L. Ramos, C. Pimentel, R. Gagliardi, W. L. Sanvito, N. Lander & R. Mechoulam, (1980) Chronic administration of cannabidiol to healthy volunteers and epileptic patients. Pharmacology 21: 175-185.
Eisohly, H. N., C. E. Turner, A. M. Clark & M. A. Eisohly, (1982) Synthesis and antimicrobial activities of certain cannabichromene and cannabigerol related compounds. J Pharm Sci 71: 1319-1323.
Fang, L., Li, X., Li, L., Li, S., Liao, X., Sun, J. and Liu, Y., 2016, “Co-spread of metal and antibiotic resistance within ST3-IncHI2 plasmids from E. coli isolates of food-producing animals,” Scientific reports, 6(1), pp. 1-8.
Farha, M. A., El-Halfawy, O. M., Gale, R. T., Macnair, C. R., Carfrae, L. A., Zhang, X., Jentsch, N. G., Magolan, J., and Brown, E. D., 2020, “Uncovering the Hidden Antibiotic Potential of Cannabis,” ACS Infect. Dis., 6(3), pp. 338-346.
Finley, P. J., Norton, R., Austin, C., Mitchell, A., Zank, S., Durham, P., 2015, “Unprecedented silver resistance in clinically isolated Enterobacteriaceae: major implications for burn and wound management,” Antimicrob Agents Chemother, 59(8), pp. 4734-4741.
Franci, G., Falanga, A., Galdiero, S., Palomba, L., Rai, M., Morelli, G., and Galdiero, M., 2015, “Silver Nanoparticles as Potential Antibacterial Agents,” Molecules, 20(5), pp. 8856-8874.
Galloway-Pena, J. R., S. R. Nallapareddy, C. A. Arias, G. M. Eliopoulos & B. E. Murray, (2009) Analysis of clonality and antibiotic resistance among early clinical isolates of Enterococcus faecium in the United States. J Infect Dis 200: 1566-1573.
Gemmell, C. G., Edwards, D. I., Fraise, A. P., Gould, F. K., Ridgway, G. L., and Warren, R. E., 2006, “Guidelines for the prophylaxis and treatment of methicillin-resistant Staphylococcus aureus (MRSA) infections in the UK,” Journal of Antimicrobial Chemotherapy, 57(4), pp. 589-608.
Gupta, A., Matsui, K., Lo, J. F. and Silver, S., 1999, “Molecular basis for resistance to silver cations in Salmonella,” Nature medicine, 5(2), pp. 183-188.
Hanczvikkel, A., Füzi, M., Ungvari, E. and Tóth, Á., 2018, “Transmissible silver resistance readily evolves in high-risk clone isolates of Klebsiella pneumoniae,” Acta microbiologica et immunologica Hungarica, 65(3), pp. 387-403.
Hidron, A. I., J. R. Edwards, J. Patel, T. C. Horan, D. M. Sievert, D. A. Pollock, S. K. Fridkin, T. National Healthcare Safety Network & F. Participating National Healthcare Safety Network, (2008) NHSN annual update: antimicrobial-resistant pathogens associated with healthcare-associated infections: annual summary of data reported to the National Healthcare Safety Network at the Centers for Disease Control and Prevention, 2006-2007. Infect Control Hosp Epidemiol 29: 996-1011.
Hosny, A. E. D. M. S., Rasmy, S. A., Aboul-Magd, D. S., Kashef, M. T., El-Bazza, Z. E., 2019, “The increasing threat of silver-resistance in clinical isolates from wounds and burns,” Infect. Drug Resist., 12, pp. 1985-2001.
Hwang, I. sok, Hwang, J. H., Choi, H., Kim, K. J., and Lee, D. G., 2012, “Synergistic Effects between Silver Nanoparticles and Antibiotics and the Mechanisms Involved,” J. Med. Microbiol., 61 (PART12), pp. 1719-1726.
Iseppi, R., Brighenti, V., Licata, M., Lambertini, A., Sabia, C., Messi, P., Pellati, F., and Benvenuti, S., 2019, “Chemical Characterization and Evaluation of the Antibacterial Activity of Essential Oils from Fibre-Type Cannabis Sativa L. (Hemp),” Molecules, 24(12), pp. 7-12.
Joung, D. K., Kang, 0. H., Seo, Y. S., Zhou, T., Lee, Y. S., Han, S. H., Mun, S. H., Kong, R., Song, H. J., Shin, D. W., and Kwon, D. Y., 2016, “Luteolin Potentiates the Effects of Aminoglycoside and β-Lactam Antibiotics against Methicillin-Resistant Staphylococcus Aureus in Vitro,” Exp. Ther. Med., 11(6), pp. 2597-2601.
Khundkar, R., Malic, C., and Burge, T., 2010, “Use of Acticoat™ Dressings in Burns: What Is the Evidence?,” Burns, 36(6), pp. 751-758.
Kosgodage, U. S., Matewele, P., Awamaria, B., Kraev, I., Warde, P., Mastroianni, G., Nunn, A. V., Guy, G. W., Bell, J. D., Inal, J. M., and Lange, S., 2019, “Cannabidiol Is a Novel Modulator of Bacterial Membrane Vesicles,” Front. Cell. Infect. Microbiol., 9 (September), pp. 1-13.
Lam, T. B. L., Omar, M. I., Fisher, E., Gillies, K., MacLennan, S., 2014, “Types of indwelling urethral catheters for short-term catheterization in hospitalised adults,” Cochrane Database of Systematic Reviews, (9).
Lok, C. N., Ho, C. M., Chen, R., Tam, P. K. H., Chiu, J. F., Che, C. M., 2008, “Proteomic identification of the Cus system as a major determinant of constitutive Escherichia coli silver resistance of chromosomal origin,” Journal of proteome research, 7(6), pp. 2351-2356.
Luo X, Reiter M A, d'Espaux L, Wong J, Denby C M, Lechner A, Zhang Y, Grzybowski A T, Harth S, Lin W, Lee H, Yu C, Shin J, Deng K, Benites V T, Wang G, Baidoo E E K, Chen Y, Dev I, Petzold C J, Keasling J D. 2019. Complete biosynthesis of cannabinoids and their unnatural analogues in yeast. Nature 567:123-126
Massani, M. B., Klumpp, J., Widmer, M., Speck, C., Nisple, M., Lehmann, R. and Schuppler, M., 2018, “Chromosomal Sil system contributes to silver resistance in E. coli ATCC 8739,” Biometals, 31(6), pp. 1101-1114.
McHugh, G. L., Moellering, R. C., Hopkins, C. C., Swartz, M. N., 1975, “Salmonella typhimurium resistant to silver nitrate, chloramphenicol, and ampicillin,” Lancet, 305(7901), pp. 235-240.
Mechoulam, R. & Y. Gaoni, (1965) Hashish. IV. The isolation and structure of cannabinolic cannabidiolic and cannabigerolic acids. Tetrahedron 21: 1223-1229.
Miao V, Coëffet-Le Gal M F, Nguyen K, Brian P, Penn J, Whiting A, Steele J, Kau D, Martin S, Ford R, Gibson T, Bouchard M, Wrigley S K, Baltz R H (March 2006). “Genetic engineering in Streptomyces roseosporus to produce hybrid lipopeptide antibiotics”. Chemistry & Biology. 13 (3): 269-76.
Morales, P., D. P. Hurst & P. H. Reggio, (2017) Molecular Targets of the Phytocannabinoids: A Complex Picture. Prog Chem Org Nat Prod 103: 103-131.
Murdoch, D. R., G. R. Corey, B. Hoen, J. M. Miro, V. G. Fowler, Jr., A. S. Bayer, A. W. Karchmer, L. Olaison, P. A. Pappas, P. Moreillon, S. T. Chambers, V. H. Chu, V. Falco, D. J. Holland, P. Jones, J. L. Klein, N. J. Raymond, K. M. Read, M. F. Tripodi, R. Utili, A. Wang, C. W. Woods, C. H. Cabell & I. International Collaboration on Endocarditis-Prospective Cohort Study, (2009) Clinical presentation, etiology, and outcome of infective endocarditis in the 21st century: the International Collaboration on Endocarditis-Prospective Cohort Study. Arch Intern Med 169: 463-473.
Najafi K, Ganbarov K, Gholizadeh P, Tanomand A, Rezaee M A, Mahmood S S, Asgharzadeh M, Kafil H S. 2019. Oral cavity infection by Enterococcus faecalis: virulence factors and pathogenesis. Reviews in Medical Microbiology 29: OOO Publish Ahead of Print.
Navarro, G., Varani, K., Reyes-Resina, I., de Medina, V. S., Rivas-Santisteban, R., Callado, C. S. C., Vincenzi, F., Casano, S., Ferreiro-Vera, C., Canela, E. I., Borea, P. A., Nadal, X., and Franco, R., 2018, “Cannabigerol Action at Cannabinoid CB1 and CB2 Receptors and at CB1-CB2 Heteroreceptor Complexes,” Front. Pharmacol., 9, pp. 1-14.
Nguyen K T, Kau D, Gu J Q, Brian P, Wrigley S K, Baltz R H, Miao V (September 2006). “A glutamic acid 3-methyltransferase encoded by an accessory gene locus important for daptomycin biosynthesis in Streptomyces roseosporus”. Molecular Microbiology. 61 (5): 1294-307.
Nguyen, K. T., He, X., Alexander, D. C., Li, C., Gu, J. Q., Mascio, C., Van Praagh, A., Mortin, L., Chu, M., Silverman, J. A., Brian, P., & Baltz, R. H. (2010). Genetically engineered lipopeptide antibiotics related to A54145 and daptomycin with improved properties. Antimicrobial agents and chemotherapy, 54(4), 1404-1413.
Odds, F. C., Synergy, antagonism, and what the chequerboard puts between them, Journal of Antimicrobial Chemotherapy, (2003) 52, 1.
PACHER, P., BA'TKAI, S., and KUNOS, G., 2006, “The Endocannabinoid System as an Emerging Target of Pharmacotherapy,” Pharmacol. Rev., 58(1), pp. 389-462.
Paganelli, Fernanda L et al. “Enterococcus faecium biofilm formation: identification of major autolysin AtIAEfm, associated Acm surface localization, and AtIAEfm-independent extracellular DNA Release.” mBio vol. 4,2 e00154. 16 Apr. 2013, doi:10.1128/mBio.00154-13.
Pertwee, R. G., 2008, “The Diverse CB 1 and CB 2 Receptor Pharmacology of Three Plant Cannabinoids: Δ 9-Tetrahydrocannabinol, Cannabidiol and Δ 9-Tetrahydrocannabivarin,” Br. J. Pharmacol., 153(2), pp. 199-215.
Pinzi, L., Lherbet, C., Baltas, M., Pellati, F., and Rastelli, G., 2019, “In Silico Repositioning of Cannabigerol as a Novel Inhibitor of the Enoyl Acyl Carrier Protein (ACP) Reductase (INHA),” Molecules, 24(14).
Prematunge, C., C. MacDougall, J. Johnstone, K. Adomako, F. Lam, J. Robertson & G. Garber, (2016) VRE and VSE Bacteremia Outcomes in the Era of Effective VRE Therapy: A Systematic Review and Meta-analysis. Infect Control Hosp Epidemiol 37: 26-35.
Rai, M., Yadav, A., and Gade, A., 2009, “Silver Nanoparticles as a New Generation of Antimicrobials,” Biotechnol. Adv., 27(1), pp. 76-83.
Rakholiya, K. D., Kaneria, M. J., and Chanda, S. V., 2013, “Medicinal Plants as Alternative Sources of Therapeutics against Multidrug-Resistant Pathogenic Microorganisms Based on Their Antimicrobial Potential and Synergistic Properties,” Fighting Multidrug Resistance with Herbal Extracts, Essential Oils and Their Components, Academic Press, pp. 165-179.
Ruben Morones-Ramirez, J., Winkler, J. A., Spina, C. S., and Collins, J. J., 2013, “Silver Enhances Antibiotic Activity against Gram-Negative Bacteria,” Sci. Transl. Med., 5(190), pp. 1-12.
Russo, E. B., (2011) Taming THC: potential cannabis synergy and phytocannabinoid-terpenoid entourage effects. Br 0.1 Pharmacol 163: 1344-1364.
Staehlin, B. M., Gibbons, J. G., Rokas, A., O'Halloran, T. V. and Slot, J. C., 2016, “Evolution of a heavy metal homeostasis/resistance island reflects increasing copper stress in enterobacteria,” Genome biology and evolution, 8(3), pp. 811-826.
Stahl, V., and Vasudevan, K., 2020, “Comparison of Efficacy of Cannabinoids versus Commercial Oral Care Products in Reducing Bacterial Content from Dental Plaque: A Preliminary Observation,” Cureus, 12(1), pp. 1-12.
Storm-Versloot, M. N., Vos, C. G., Ubbink, D. T. and Vermeulen, H., 2010, “Topical silver for preventing wound infection,” Cochrane Database of Systematic Reviews, (3).
Sutterlin, S., Edquist, P., Sandegren, L., Adler, M., Tangden, T., Drobni, M., Olsen, B. and Melhus, Å., 2014, “Silver resistance genes are overrepresented among Escherichia coli isolates with CTX-M production,” Appl. Environ. Microbiol., 80(22), pp. 6863-6869.
Trost and Dogra, (2007) Synthesis of (-)-Δ9-trans-Tetrahydrocannabinol Stereocontrol via Mo-catalyzed Asymmetric Allylic Alkylation Reaction. Org Lett. 2007 Mar. 1; 9(5): 861-863.
Turner, C. E. & M. A. Elsohly, (1981) Biological activity of cannabichromene, its homologs and isomers. J Clin Pharmacol 21: 2835-2915.
Van Klingeren, B. & M. Ten Ham, (1976) Antibacterial activity of delta9-tetrahydrocannabinol and cannabidiol. Antonie Van Leeuwenhoek 42: 9-12.
Udoh, M., Santiago, M., Devenish, S., McGregor, I. S., and Connor, M., 2019, “Cannabichromene Is a Cannabinoid CB2 Receptor Agonist,” Br. J. Pharmacol., 176(23), pp. 4537-4547.
Vermeulen, H., van Hattem, J. M., Storm-Versloot, M. N., Ubbink, D. T. and Westerbos, S. J., 2007, “Topical silver for treating infected wounds,” Cochrane Database of Systematic Reviews, (1).
Zhong et al., (2017) Comparative genomic analysis of the genus Enterococcus. Microbiological Research, Volume 196, March 2017, Pages 95-105.

Citation of references herein is not an admission that such references are prior art to the present invention. Any priority document(s) and all publications, including but not limited to patents and patent applications, cited in this specification are incorporated herein by reference. All documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual publication were specifically and individually indicated to be incorporated by reference herein and as though fully set forth herein. The invention includes all embodiments and variations substantially as hereinbefore described and with reference to the examples and drawings. In some embodiments, the invention excludes steps that involve medical or surgical treatment.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing.

1. A method of treating or preventing a bacterial infection in a subject in need thereof, comprising administering to the subject an effective amount of:
a cannabinoid that is one or more of cannabidiol (CBD), cannabidiolic acid (CBDA), cannabigerol (CBG), cannabigerolic acid (CBGA), cannabichromenic acid (CBCA) or cannabichromene (CBC); and,
a silver-containing medicament;
wherein the cannabinoid and the silver-containing medicament are each administered in a regimen, and the combination of the regimens provides a positive drug-drug interaction between the cannabinoid and the silver-containing medicament in the subject.

2. The method of claim 1, wherein the positive drug-drug interaction between the cannabinoid and the silver-containing medicament is a positive antibiotic drug-drug interaction that enhances the antibiotic effect of the cannabinoid and/or the silver-containing medicament in the subject.

3. The method of claim 1 or 2, wherein the positive drug-drug antibiotic interaction comprises a synergistically effective combined antibiotic activity.

4. The method of any one of claims 1 to 3, wherein the bacterial infection comprises infection by a gram positive bacteria.

5. The method of any one of claims 1 to 4, wherein the bacterial infection comprises infection by a plurality of gram positive bacteria.

6. The method of any one of claims 1 to 5, wherein the bacterial infection comprises infection by a gram negative bacteria.

7. The method of any one of claims 1 to 6, wherein the bacterial infection comprises infection by a plurality of gram negative bacteria.

8. The method of any one of claims 1 to 7, wherein the bacterial infection comprises infection by an antibiotic resistant bacteria.

9. The method of any one of claims 1 to 8, wherein the cannabinoid is administered in a regimen that reduces the minimum inhibitory concentration (MIC) of the silver-containing medicament.

10. The method of claim 9, wherein the cannabinoid reduces the MIC of the silver-containing medicament when the cannabinoid is present in an amount that is less than the MIC of the cannabinoid.

11. The method of any one of claims 1 to 10, wherein the silver-containing medicament is administered in a regimen that reduces the minimum inhibitory concentration (MIC) of the cannabinoid.

12. The method of claim 11, wherein the silver-containing medicament reduces the MIC of the cannabinoid when the silver-containing medicament is present in an amount that is less than the MIC of the silver-containing medicament.

13. The method of any one of claims 1 to 12, wherein the cannabinoid is administered in a relative amount that provides at least a 2 to 128 fold decrease in minimum inhibitory concentration (MIC) of the silver-containing medicament.

14. The method of any one of claims 1 to 13, wherein the silver-containing medicament is administered in a relative amount that provides at least a 2 to 128 fold decrease in minimum inhibitory concentration (MIC) of the cannabinoid.

15. The method of any one of claims 1 to 14, wherein the cannabinoid is one of CBD, CBDA, CBG, CBGA, CBCA or CBC.

16. The method of any one of claims 1 to 14, wherein the cannabinoid is two, three or four of CBD, CBDA, CBG, CBGA, CBCA or CBC.

17. The method of any one of claims 1 to 14, wherein the cannabinoid is CBD, CBDA, CBG, CBGA, CBCA and CBC.

18. The method of any one of claims 1 to 17, wherein the cannabinoid is derived from a plant.

19. The method of claim 18, wherein the plant is a Cannabis sativa or Cannabis indica plant.

20. The method of any one of claims 1 to 19, wherein no antibiotic other than the cannabinoid and the silver-containing medicament is administered to the subject.

21. The method of any one of claims 1 to 20, wherein the method consists essentially of administering to the subject the effective amounts of the cannabinoid and the silver-containing medicament.

22. The method of any one of claims 1 to 21, wherein no phytocannabinoid other than the cannabinoid is administered to the subject.

23. The method of any one of claims 1 to 22, wherein the silver-containing medicament is one or more of: a silver salt, silver nitrate, silver sulfate, silver oxide, silver chloride, silver lactate, a silver nanoparticle, a colloidal silver, a silver zeolite, or silver sulfadiazine.

24. The method of any one of claims 1 to 23, wherein the subject is a mammal.

25. The method of claim 24, wherein the subject is a human patient.

26. The method of any one of claims 1 to 25, wherein the therapeutically effective regimen of the cannabinoid comprises administration of from 0.001 to 5,000 mg per day of the cannabinoid.

27. The method of any one of claims 1 to 26, wherein the therapeutically effective regimen of the silver-containing medicament comprises administration of from 0.001 to 10,000 mg per day elemental silver of the silver-containing medicament.

28. The method of any one of claims 1 to 27, wherein the cannabinoid and the silver-containing medicament are co-administered.

29. The method of any one of claims 1 to 28, wherein the cannabinoid and the silver-containing medicament are administered sequentially, in any order.

30. An antibiotic formulation, comprising:
a cannabinoid that is one or more of cannabidiol (CBD), cannabidiolic acid (CBDA), cannabigerol (CBG), cannabigerolic acid (CBGA), cannabichromenic acid (CBCA) or cannabichromene (CBC); and,
a silver-containing medicament;
wherein the cannabinoid and the silver-containing medicament are each present in an amount, and the combination of the amounts provides a positive drug-drug interaction between the cannabinoid and the silver-containing medicament in a subject when the formulation is administered to the subject.

31. The antibiotic formulation of claim 30, wherein the cannabinoid is present in the formulation at 0.01-5% w/w.

32. The antibiotic formulation of claim 30 or 31, wherein the silver-containing medicament is present in the formulation at 0.01-5% w/w.

33. The antibiotic formulation of any one of claims 30 to 32, wherein the cannabinoid and/or the silver-containing medicament are dissolved, dispersed, mixed or suspended in the formulation with a pharmaceutically acceptable carrier.

34. The antibiotic formulation of any one of claims 30 to 33, wherein the positive drug-drug interaction between the cannabinoid and the silver-containing medicament is a positive antibiotic drug-drug interaction that enhances the antibiotic effect of the cannabinoid and/or the silver-containing medicament in the subject.

35. The antibiotic formulation of any one of claims 30 to 34, wherein the positive drug-drug antibiotic interaction comprises a synergistically effective combined antibiotic activity.

36. The antibiotic formulation of any one of claims 30 to 35, wherein the cannabinoid is one of CBD, CBDA, CBG, CBGA, CBCA or CBC.

37. The antibiotic formulation of any one of claims 30 to 35, wherein the cannabinoid is two, three or four of CBD, CBDA, CBG, CBGA, CBCA or CBC.

38. The antibiotic formulation of any one of claims 30 to 35, wherein the cannabinoid is CBD, CBDA, CBG, CBGA, CBCA and CBC.

39. The antibiotic formulation of any one of claims 30 to 38, wherein the cannabinoid is derived from a plant.

40. The antibiotic formulation of claim 39, wherein the plant is a Cannabis sativa or Cannabis indica plant.

41. The antibiotic formulation of any one of claims 30 to 40, wherein no antibiotic other than the cannabinoid and the silver-containing medicament is present in the formulation.

42. The antibiotic formulation of any one of claims 30 to 41, wherein the formulation consists essentially of the cannabinoid and the silver-containing medicament as active ingredients.

43. The antibiotic formulation of any one of claims 30 to 42, wherein the formulation comprises no phytocannabinoid other than the cannabinoid.

44. The antibiotic formulation of any one of claims 30 to 43, wherein the silver-containing medicament is one or more of: a silver salt, silver nitrate, silver sulfate, silver oxide, silver chloride, silver lactate, a silver nanoparticle, a colloidal silver, a silver zeolite, or silver sulfadiazine.

45. The antibiotic formulation of any one of claims 30 to 44, wherein the antibiotic formulation is for use in formulating a medicament for treating a bacterial infection in a subject in need thereof.

46. The antibiotic formulation of claim 45, wherein the bacterial infection comprises infection by a gram positive bacteria.

47. The antibiotic formulation of claim 45 or 46, wherein the bacterial infection comprises infection by a plurality of gram positive bacteria.

48. The antibiotic formulation of any one of claims 45 to 47, wherein the bacterial infection comprises infection by a gram negative bacteria.

49. The antibiotic formulation of any one of claims 45 to 48, wherein the bacterial infection comprises infection by a plurality of gram negative bacteria.

50. The antibiotic formulation of any one of claims 45 to 49, wherein the bacterial infection comprises infection by an antibiotic resistant bacteria.

51. The antibiotic formulation of any one of claims 45 to 50, wherein the cannabinoid is administered in a regimen that reduces the minimum inhibitory concentration (MIC) of the silver-containing medicament.

52. The antibiotic formulation of claim 51, wherein the cannabinoid reduces the MIC of the silver-containing medicament when the cannabinoid is present in an amount that is less than the MIC of the cannabinoid.

53. The antibiotic formulation of any one of claims 45 to 52, wherein the silver-containing medicament is administered in a regimen that reduces the minimum inhibitory concentration (MIC) of the cannabinoid.

54. The antibiotic formulation of claim 53, wherein the silver-containing medicament reduces the MIC of the cannabinoid when the silver-containing medicament is present in an amount that is less than the MIC of the silver-containing medicament.

55. The antibiotic formulation of any one of claims 45 to 54, wherein the cannabinoid is administered in a relative amount that provides at least a 2 to 128 fold decrease in minimum inhibitory concentration (MIC) of the silver-containing medicament.

56. The antibiotic formulation of any one of claims 45 to 55, wherein the silver-containing medicament is administered in a relative amount that provides at least a 2 to 128 fold decrease in minimum inhibitory concentration (MIC) of the cannabinoid.

57. The antibiotic formulation of any one of claims 45 to 56, wherein the formulation comprises more than one silver-containing medicament.

58. The antibiotic formulation of any one of claims 30 to 57, wherein the antibiotic formulation is provided in or coating a supporting matrix.

59. The antibiotic formulation of claim 58, wherein the supporting matrix comprises a gel, a hydrogel, a film, a polymer or a ceramic.

60. The antibiotic formulation of claim 58 or 59, wherein the supporting matrix is in the form of a wound dressing, a biomedical implant, a periodontal or endodontal device, an endotracheal tube, a surgical mask, cotton fibers, synthetic fibers, a component of an invasive medical device, a catheter or a catheter coating.

61. The antibiotic formulation of claim 59 or 60, wherein the antibiotic formulation comprises a hydrogel formulation that is a dried film.

62. The antibiotic formulation of claim 61, wherein the dried film is in the form of, or is for use as, a wound dressing.

63. The antibiotic formulation of claim 61 or 62, wherein the hydrogel material is poly vinyl alcohol.

64. The antibiotic formulation of ay one of claims 61 to 63, wherein the hydrogel formulation coats a catheter.

65. The antibiotic formulation of claim 64, where the catheter is a urethral catheter.

66. The antibiotic formulation of any one of claims 58 to 65, wherein the matrix comprises more than one silver-containing medicament and the releasability of the different silver-containing medicaments from the matrix is different.

67. The antibiotic formulation of claim 66, wherein a first silver-containing medicament is formulated in the matrix for sustained release, and a second silver-containing medicament is formulated in the matrix for quick release.

68. The antibiotic formulation of any one of claims 58 to 65, wherein the matrix comprises an additional medicament, and the releasability from the matrix of the additional medicament is different from the releasability of the silver-containing medicament.

69. The antibiotic formulation of claim 68, wherein the additional medicament is an additional antibiotic.

70. Use of the antibiotic formulation of any one of claims 30 to 69, for treating or preventing a bacterial infection in a subject in need thereof.

71. Use of one or more antibiotic formulations for treating or preventing a bacterial infection in a subject in need thereof, wherein the use is of an effective amount of:
a cannabinoid that is one or more of cannabidiol (CBD), cannabidiolic acid (CBDA), cannabigerol (CBG), cannabigerolic acid (CBGA), cannabichromenic acid (CBCA) or cannabichromene (CBC); and,
a silver-containing medicament;
wherein the cannabinoid and the silver-containing medicament are each for use in a regimen, and the combination of the regimens provides a positive drug-drug interaction between the cannabinoid and the silver-containing medicament in the subject.

72. The use according to claim 71, wherein the positive drug-drug interaction between the cannabinoid and the silver-containing medicament is a positive antibiotic drug-drug interaction that enhances the antibiotic effect of the cannabinoid and/or the silver-containing medicament in the subject.

73. The use according to claim 71 or 72, wherein the positive drug-drug antibiotic interaction comprises a synergistically effective combined antibiotic activity.

74. The use according to any one of claims 71 to 73, wherein the bacterial infection comprises infection by a gram positive and/or gram negative bacteria.

75. The use according to any one of claims 71 to 74, wherein the bacterial infection comprises infection by a plurality of gram positive and/or gram negative bacteria.

76. The use according to any one of claims 71 to 75, wherein the bacterial infection comprises infection by an antibiotic resistant bacteria.

77. The use according to any one of claims 71 to 76, wherein the cannabinoid is for use in a regimen that reduces the minimum inhibitory concentration (MIC) of the silver-containing medicament.

78. The use according to claim 77, wherein the cannabinoid reduces the MIC of the silver-containing medicament when the cannabinoid is present in an amount that is less than the MIC of the cannabinoid.

79. The use according to any one of claims 71 to 78, wherein the silver-containing medicament is for use in a regimen that reduces the minimum inhibitory concentration (MIC) of the cannabinoid.

80. The use according to claim 79, wherein the silver-containing medicament reduces the MIC of the cannabinoid when the silver-containing medicament is present in an amount that is less than the MIC of the silver-containing medicament.

81. The use according to any one of claims 71 to 80, wherein the cannabinoid is for use in a relative amount that provides at least a 2 to 128 fold decrease in minimum inhibitory concentration (MIC) of the silver-containing medicament.

82. The use according to any one of claims 71 to 81, wherein the silver-containing medicament is for use in a relative amount that provides at least a 2 to 128 fold decrease in minimum inhibitory concentration (MIC) of the cannabinoid.

83. The use according to any one of claims 71 to 82, wherein the cannabinoid is one of CBD, CBDA, CBG, CBGA, CBCA or CBC.

84. The use according to any one of claims 71 to 82, wherein the cannabinoid is two, three or four of CBD, CBDA, CBG, CBGA, CBCA or CBC.

85. The use according to any one of claims 71 to 82, wherein the cannabinoid is CBD, CBDA, CBG, CBGA, CBCA and CBC.

86. The use according to any one of claims 71 to 85, wherein the cannabinoid is derived from a plant.

87. The use according to claim 86, wherein the plant is a Cannabis sativa or Cannabis indica plant.

88. The use according to any one of claims 71 to 87, wherein no antibiotic other than the cannabinoid and the silver-containing medicament is used to the subject.

89. The use according to any one of claims 71 to 88, wherein the method use consists essentially of use in the subject of the effective amounts of the cannabinoid and the silver-containing medicament.

90. The use according to any one of claims 71 to 89, wherein no phytocannabinoid other than the cannabinoid is administered to the subject.

91. The use according to any one of claims 71 to 90, wherein the silver-containing medicament is one or more of: a silver salt, silver nitrate, silver sulfate, silver oxide, silver chloride, silver lactate, a silver nanoparticle, a colloidal silver, a silver zeolite, or silver sulfadiazine.

92. The use according to any one of claims 70 to 91, wherein the subject is a mammal.

93. The use according to claim 92, wherein the subject is a human patient.

94. The use according to any one of claims 70 to 93, wherein the therapeutically effective regimen of the cannabinoid comprises use of from 0.001 to 5,000 mg per day of the cannabinoid.

95. The use according to any one of claims 70 to 94, wherein the therapeutically effective regimen of the silver-containing medicament comprises administration of from 0.001 to 10,000 mg per day elemental silver of the silver-containing medicament.

96. The use according to any one of claims 70 to 95, wherein the cannabinoid and the silver-containing medicament are for co-administration.

97. The use according to any one of claims 70 to 96, wherein the cannabinoid and the silver-containing medicament are for use sequentially, in any order.
